SEQUENCE LISTING (28 sequences)

SEQ ID NO: 1 - DNA, 27 bp, synthetic construct
  source 1..27: mol_type = other DNA
ttacatgtgactgtgttcagggtatag

SEQ ID NO: 2 - DNA, 25 bp, synthetic construct
  source 1..25: mol_type = other DNA
catagctagccgtacatccacatct

SEQ ID NO: 3 - DNA, 30 bp, synthetic construct
  source 1..30: mol_type = other DNA
ttagcggccgcgtacggctttagaaatgag

SEQ ID NO: 4 - DNA, 27 bp, synthetic construct
  source 1..27: mol_type = other DNA
ttcacgtagtgggtctgaactgggcac

SEQ ID NO: 5 - DNA, 30 bp, synthetic construct
  source 1..30: mol_type = other DNA
ttacatgtggtgcagccaatactattacag

SEQ ID NO: 6 - DNA, 25 bp, synthetic construct
  source 1..25: mol_type = other DNA
atagctagcatgaaggctgaacgtg

SEQ ID NO: 7 - DNA, 29 bp, synthetic construct
  source 1..29: mol_type = other DNA
tagcggccgcgtcttatagcatgaattcg

SEQ ID NO: 8 - DNA, 28 bp, synthetic construct
  source 1..28: mol_type = other DNA
ttcacgtagtgttgcgagcattactgcg

SEQ ID NO: 9 - DNA, 31 bp, synthetic construct
  source 1..31: mol_type = other DNA
tcacgtagtggaataccgacggcgttaatag

SEQ ID NO: 10 - DNA, 32 bp, synthetic construct
  source 1..32: mol_type = other DNA
ttgcggccgcatcatgaatgcgtataataaag

SEQ ID NO: 11 - DNA, 35 bp, synthetic construct
  source 1..35: mol_type = other DNA
tagctagcgataaacttaatgaaaaatgtttttcg

SEQ ID NO: 12 - DNA, 24 bp, synthetic construct
  source 1..24: mol_type = other DNA
ttacatgtcctgtacgatgagttc

SEQ ID NO: 13 - DNA, 22 bp, synthetic construct
  source 1..22: mol_type = other DNA
gtgctataacgcgactatctag

SEQ ID NO: 14 - DNA, 20 bp, synthetic construct
  source 1..20: mol_type = other DNA
tgttggtagttcttccgtgg

SEQ ID NO: 15 - DNA, 23 bp, synthetic construct
  source 1..23: mol_type = other DNA
tttggtaatggtttctcatgtgg

SEQ ID NO: 16 - DNA, 23 bp, synthetic construct
  source 1..23: mol_type = other DNA
gacatttagtttgagtgttcctg

SEQ ID NO: 17 - DNA, 552 bp, synthetic construct
  source 1..552: mol_type = other DNA
atggcccccgcactctaccattatttccccgtacctatggatgaccattcagagaaagtcgtgttgaggaaccgtaagatgctgaagtggttatggatactgcctgtgttagctttggtgttaagtctcctggtggctctcatagtctttgctattaaagccaattctaaagcctgtaaggatggactgcttgctgaacagaagtgtcttaacaaaactaggctgttagaacttcagctgacccaggctcaagaatctttggtggtggctgaagctcaagcatacctgtgtaatcagactgtaggcaccctgaaaagctctctggaaatggagaaggatgagtctcagaaacaaagggaactggctcagaaactgcagggagaaaacggtaatctgaaacaggaactggaaaacatgatggccgagcttgagcagctgcggaaagaacacgcctcggatgaaaagaacggctctactagctctaggaatgcaaggagtttcttagttgtggcagtcttactcagcctgtcttttggcgccctactcgcatga

SEQ ID NO: 18 - DNA, 190 bp, synthetic construct
  source 1..190: mol_type = other DNA
taaaaattgaaaataaatacaaaggttcttgagggttgtgttaaattgaaagcgagaaataatcataaataggatctatccctatcagtgatagagatctccctatcagtgatagagatcagatctcgagctcaacccgggaactctgcagtcgacggtacaggtcgccaccggatcctgttaacccatg

SEQ ID NO: 19 - DNA, 159 bp, synthetic construct
  source 1..159: mol_type = other DNA
aaaaattaaaattttattttttttttttggaatataaataagatctatccctatcagtgatagagatctccctatcagtgatagagatcagatctcgagctcaacccgggaactctgcagtcgacggtacaggtcgccaccggatcctgttaacccatg

SEQ ID NO: 20 - DNA, 21 bp, synthetic construct
  source 1..21: mol_type = other DNA
ttctaatggaggctcaacaag

SEQ ID NO: 21 - DNA, 35 bp, synthetic construct
  source 1..35: mol_type = other DNA
acatagatctcccagactaagagctatttttaaac

SEQ ID NO: 22 - DNA, 35 bp, synthetic construct
  source 1..35: mol_type = other DNA
tatagcggccgcatatgctctagatactgcaaaac

SEQ ID NO: 23 - DNA, 35 bp, synthetic construct
  source 1..35: mol_type = other DNA
ttaacacgtagtgttagcatcccatggtaaatgtc

SEQ ID NO: 24 - DNA, 18 bp, synthetic construct
  source 1..18: mol_type = other DNA
gtctaacggcttcaatcg

SEQ ID NO: 25 - DNA, 18 bp, synthetic construct
  source 1..18: mol_type = other DNA
gtccggttggaaaacctt

SEQ ID NO: 26 - DNA, 20 bp, synthetic construct
  source 1..20: mol_type = other DNA
ctctctagctaccaccgcaa

SEQ ID NO: 27 - DNA, 23 bp, synthetic construct
  source 1..23: mol_type = other DNA
acggtttatctaacgacacaaca

SEQ ID NO: 28 - DNA, 681 bp, synthetic construct
  source 1..681: mol_type = other DNA
ctgtacaagggcgccatgacgtctacattttaccactacatccccctaccaatgtctgagaactcccgcgagcttatgttggaataccataaactcccgagatggctgggtatcctcctagcgctcctggtgcttctggtagtggggctgcttgtggctacgattatcctggccgtgcaggctcattcacctgcctgcaaggacggtcatcgggctgagcaggaatgcaggaactttactcacttattagagagtcaaagaaccagagcacaggagatcctcttaaaaactaaagcccaggctgccacatataatcagacggtggtcactttgatggcatcgctgaaagtagagcaggcacagggacagaagctgcgcgaacaagtgcaggaactgcaagaagagatcaagactcttaagcaaaaattacaggacacaacacagaaattgcaggtgaccacgaccgaactacaaaacactactcaaaagctccaggttaccacagctaagctccaggtgactacacaaaaactccaagatacgactaccgaactgaatcagctgcggaaggaccacgagagctttgacaggggtaacgggagcaccagctctgggaatactctgtccctgtccgtggtggtcctgcttctcacgctttcccttttggacttgctggcatga

(19) United States
(12) Patent Application Publication
(10) Pub. No.: US 2026/0308627 A1
(43) Pub. Date: Oct. 8, 2026

(54) RAPID SELECTION SYSTEM FOR THE GENERATION OF RECOMBINANT ENVELOPED VIRUSES

(71) Applicant: PROBIOGEN AG, Berlin (DE)

(72) Inventors: Ingo JORDAN, Berlin (DE); Sirine ABIDI, Berlin (DE); Nicole SEEHASE, Rheinstetten (DE); Volker SANDIG, Berlin (DE); Alexander KARLAS, Velten (DE)

(21) Appl. No.: 19/485,664

(22) PCT Filed: May 13, 2024
(86) PCT No.: PCT/EP2024/063026

§ 371 Date: Nov. 18, 2025

(30) Foreign Application Priority Data
May 26, 2023 (EP) 23175634.7
Jun. 21, 2023 (EP) 23180591.2

(51) Int. Cl. C12N 15/86; C07K 14/705

(52) U.S. Cl. CPC C12N 15/86; C07K 14/70596; C12N 2710/24143; C12N 2710/24151

(57) ABSTRACT

The present invention relates to a rapid selection system for the generation of recombinant enveloped viruses.

### CROSS REFERENCE TO RELATED APPLICATIONS

This application is a U.S. National Stage of PCT/EP2024/063026, international filing date May 13, 2024, which claims priority to European Application No. 23175634.7, filing date May 26, 2023 and European Application No. 23180591.2, filing date Jun. 21, 2023.

The present invention relates to a rapid selection system for the generation of recombinant enveloped viruses.

### REFERENCE TO SEQUENCE LISTING IN ELECTRONIC FORMAT

The contents of the electronic sequence listing (513-43-pct-sequence-listing.xml; Size: 27,332 bytes; and Date of Creation: Nov. 14, 2025) is herein incorporated by reference in its entirety.

### BACKGROUND OF THE INVENTION

The Poxviridae are a diverse family with insects and vertebrates as hosts. They are large DNA viruses that replicate in the cytoplasm without a nuclear phase (a property that defines the phylum of Nucleocytoviricota and is shared with the Asfarviridae). The fully matured enveloped virions resemble flattened barrels with up to 300 nm in length and contain a genome of 128 to 365 kb that code for more than 200 genes. Poxviruses are studied as vectors for vaccination and oncotherapy due to a unique combination of virological properties. Attenuated poxviruses have been shown to be safe for recipients and able to rapidly induce a lasting and comprehensive immune response. Recombinant poxviral vectors can carry and stably maintain expression cassettes also for very large transgenes at different positions within the genome. Because poxviruses replicate without a nuclear phase risk of chromosomal aberrations is low in case of an abortive infection.

However, multiplication in the cytoplasm also complicates genetic manipulation because cellular enzymes that interpret and replicate DNA are not localized in this compartment. For this reason, infection with helper virus is required (if a complete viral genome is provided as recombinant DNA) or infection with a receiving virus (if a smaller shuttle DNA is provided) to obtain the desired recombinant vectors. Helper viruses provide the viral replication machinery that allows rescue of recombinant viruses out of ectopic genomic DNA. Because of the large size of poxviruses and asfarviruses, a complete genome can only be accommodated by artificial chromosomes. Because conventional plasmids can be manipulated more easily, the commonly used method to obtain recombinant viruses is based on transfection of shuttle plasmids into cells infected with the receiving virus. The shuttle plasmids contain the gene of interest flanked by regions homologous to a segment of the viral genome. Recombination of shuttle plasmid and receiving virus yield the desired vectors. Both protocols require removal of parental (original) viruses via cumbersome purification processes so that a pure preparation of recombinants is obtained. The limitations of the current processes are compared and described in a recent publication (especially FIGS. 1 and 2 in (Kugler et al. 2019)).

For homologous recombination, one challenge is that very few recombinant viruses are generated and that these are difficult to identify in the adherent cell culture monolayers. The few recombinants must be identified, marked and isolated (picked) by experienced operators with pipettes that directly access the infected monolayer. The isolated plaques are resuspended in medium and transferred to a fresh monolayer. Again, usually after 48-72 h, recombinant plaques must be identified, isolated and again transferred. This process is repeated until parental virus has been completely removed. Plaque picking is associated with a risk for contamination because of the additional manipulation (pipet tips, agarose overlay, animal sera in the adherent culture medium) and from internal sources (neighboring plaques with parental virus). The fitness of recombinant viruses can be lower compared to the parental virus depending on the desired transgenes that are being expressed. This provides a replication advantage to the parental virus. Depending on the fitness of the recombinant virus between 5-8 cycles of plaque purification are required until a pure recombinant preparation is obtained.

For rescue of recombinant viruses out of bacterial artificial chromosomes (BACs) the challenges start with manipulation of the very large infectious DNA constructs. Recombination is assisted by induced marker deletion in special strains of E. coli bacteria. The BACs are purified and transfected into suitable host cells. After infection with the helper virus the recombinant progeny is formed out of the BACs, catalyzed by the replication machinery that is provided in trans by the helper virus.

Again, the challenge is to identify the desired recombinants. Identification can be facilitated by insertion of a fluorescent marker into the viruses in such a way, that the marker is framed by smaller homologous flanks (Kugler et al. 2019). The marker is deleted by internal recombination via sequential passaging until a pure and marker-free preparation is achieved. The helper virus can be a related poxvirus such as fowlpox virus or rabbit fibroma virus. If a helper virus is used that can also replicate in avian cells then separation from the desired recombinants is an additional challenge. If a helper virus is used with impaired replication in avian cells then an additional cellular substrate is required specifically for production of the helper virus. In both cases, helper viruses that are not part of the final composition need to be prepared at sufficient purity and documentation which, together with dependence on animal sera for cultivation, complicate industrial application especially under GMP regimes.

The above described processes to identify the desired recombinants are time-consuming, expensive, cumbersome, lengthy, and favor contamination.

Thus, there is an unmet need to improve and optimize recombinant virus identification or selection processes.

The present invention facilitates the derivation of recombinant large nucleocytoplasmic DNA viruses, including poxviruses, drastically. The present inventors developed a process that allows to obtain the desired recombinants free of contamination with parental virus within a single passage. Only the parental virus and a shuttle plasmid comprising the gene of interest are required. The system is suitable for rescue of recombinant viruses either in adherent cells or in suspension cells that proliferate in chemically defined medium. The system is even suitable for concurrent (simultaneous) exchange of at least two expression cassettes in different, also distant positions of the genome. One example tested here is simultaneous insertion of genes of interest into the locations for thymidine kinase (TK) and deletion site VI (DS VI) of the poxviral genome. The two insertion sites are separated by 54 kb. Prior to this rapid and parallel insertion the receiving virus was generated, rescued and amplified with different tetherins at these sites. The principle of the present invention is to equip the receiving virus with a factor that impairs the ability of the parental virus to counter innate immunity cascades of a host cell or that is derived from the innate immune system of a host cell. The immune factor occupies at least one of the intended insertion sites and is highly efficient in interfering with replication of the receiving virus. Recombination with a gene of interest removes the immune factor and allows replication only of desired recombinants. Here, the present inventors used tetherin as one example for attenuation of replication. The gene of interest can be a fluorescent marker for quantification of efficiency or any therapeutically active gene such as vaccine antigen, immune enhancer or protein for molecular replacement.

To obtain a parental virus that is highly impaired as described here, the immune factor needs to be suppressed during replication. Here, the present inventors used, as one example, a Tet-Repressor (TetR) inducible promoter in the parental virus that is silent under normal conditions in the fully permissive host cell. The TetR is constitutively expressed by the host cell and can be deactivated by addition of doxycycline. Other inducible and non-inducible systems (such as RNA interference) are conceivable. The host cell can be of a lineage that proliferates in the adherent phase in presence of animal serum, but it can also be of a lineage that proliferates in suspension in chemically defined medium for industrial processes under GMP regimes. Using the TetR system has the advantage that only a single cell line is required for production of receiving virus, generation of recombinant viruses and production of the final recombinant viruses. Addition of doxycycline is required only once, for one passage at any time after the recombination.

Attenuated viruses are viruses with compromised virulence in the intended recipient, e.g. human or animal recipient. Such a property can be achieved by adaptation of a virus to narrow temperature ranges or narrow host ranges and to other artificial replication environments, including chemically defined media. Attenuated viruses should not burden the host but stimulate its immune response. Diverse attenuated viruses for prophylactic or therapeutic purposes are available but many of them do not have the desired effect on the immune system.

Thus, there is an unmet need for new attenuated viruses having an improved effect on the immune system.

The present inventors have generated a new attenuated nucleocytoplasmic large DNA virus (NCLDV), wherein at least one essential gene has been replaced with the analogous essential gene of another NCLDV from a different virus family. Especially, the present inventors have generated a vaccinia virus, wherein the genes coding for D9 and D10 of the vaccinia virus have been replaced with the (analogous) gene coding for g5R of an African swine fever virus (ASFV). D9 and D10 are decapping enzymes in the vaccinia virus that reduce accumulation of dsRNA, a potent trigger of innate immunity, in cells of a recipient. These decapping enzymes may even preferentially attack mRNAs important for innate immunity. The new mosaic virus that contains the gene coding for g5R in the vaccinia virus context causes a delayed but greater innate immune response which is beneficial to direct the immune system against intended targets such as tumors.

For applications where coinfection with a helper virus is required (for example, to rescue a recombinant virus out genomic DNA provided as artificial chromosome), the present invention further describes an attenuation (weakening) method by replacing viral defensive genes of one nucleocytoplasmic large DNA virus (NCLDV) with those from a related NCLDV.

This replacement leads to a mosaic virus with a mismatch in the genotype that leads to impaired replication. The approach maintains the original cell line dependence so that no change of cellular substrate is required. A recombination with a gene of interest flanked by the original (cognate) viral defensive genes removes the newly introduced immune factor and promotes replication of desired recombinants.

The above described new approaches are time-efficient, inexpensive, simple, and constitute a novel barrier against contamination.

### SUMMARY OF THE INVENTION

In a first aspect, the present invention relates to an enveloped virus containing a polynucleotide comprising a/at least one nucleotide sequence encoding tetherin.

In a second aspect, the present invention relates to a cell line expressing a factor antagonizing tetherin.

In a third aspect, the present invention relates to a method for propagating an enveloped virus containing a polynucleotide comprising a/at least one nucleotide sequence encoding tetherin comprising the step of:
culturing the enveloped virus containing a polynucleotide comprising a/at least one nucleotide sequence encoding tetherin in a cell line expressing a factor antagonizing tetherin.

In a fourth aspect, the present invention relates to a method for producing a virus population comprising a recombinant enveloped virus containing a polynucleotide comprising a nucleotide sequence of interest comprising the steps of:
(i) transfecting a cell line which
   (a) expresses a factor antagonizing tetherin and
   (b) comprises an enveloped virus containing a first polynucleotide comprising a/at least one nucleotide sequence encoding tetherin
   with a second polynucleotide comprising a nucleotide sequence of interest,
   wherein the second polynucleotide is capable of homologous recombination with the first polynucleotide contained in the enveloped virus, and
(ii) culturing the enveloped virus in the cell line transfected in (i), thereby obtaining a virus population that is composed of a recombinant enveloped virus comprising, instead of the/the at least one nucleotide sequence encoding tetherin, the nucleotide sequence of interest, and an enveloped virus containing the first polynucleotide.

In a fifth aspect, the present invention relates to a pharmaceutical composition comprising an enveloped virus according to the first aspect.

In a sixth aspect, the present invention relates to an enveloped virus according to the first aspect or a pharmaceutical composition according to the sixth aspect for use in medicine.

In a seventh aspect, the present invention relates to an enveloped virus according to the first aspect or a pharmaceutical composition according to the sixth aspect for use in therapy.

In an eight aspect, the present invention relates to a nucleocytoplasmic large DNA virus (NCLDV), wherein at least one essential gene has been replaced with the analogous essential gene of another NCLDV from a different virus family.

In a ninth aspect, the present invention relates to a pharmaceutical composition comprising a nucleocytoplasmic large DNA virus (NCLDV) according to the eight aspect.

In a tenth aspect, the present invention relates to a nucleocytoplasmic large DNA virus (NCLDV) according to the eight aspect or a pharmaceutical composition according to the ninth aspect for use in medicine.

In an eleventh aspect, the present invention relates to a nucleocytoplasmic large DNA virus (NCLDV) according to the eight aspect or a pharmaceutical composition according to the ninth aspect for use in therapy.

This summary of the invention does not describe all features of the invention.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

The term “consisting essentially of”, as used herein, limits the scope of a claim to the specified materials or steps and those that do not materially affect the basic and novel characteristic(s) of the claimed invention. In other words, the term “consisting essentially of”, as used herein, is generally construed to mean that the composition or formulation (a) necessarily includes the listed ingredients and (b) is open to unlisted ingredients that do not materially affect the basic and novel properties of the composition. Similarly, when “consisting essentially of” is used herein in a process claim, the claim requires that the listed steps are performed, but also may include unlisted steps that do not affect the basic and material properties of the process.

The term “about” when used in connection with a numerical value is meant to encompass numerical values within a range having a lower limit that is 5% smaller than the indicated numerical value and having an upper limit that is 5% larger than the indicated numerical value.

The terms “polypeptide” and “protein” are used interchangeably in the context of the present invention and refer to a long peptide-linked chain of amino acids.

The term “virus”, as used herein, refers to a molecule that replicates only inside living cells of other organisms. It may also be cultivated in cell culture. Viruses can infect all types of life forms, from animals and plants to microorganisms including bacteria and archaea. While not inside an infected cell or in the process of infecting a cell, viruses exist in the form of independent particles. These viral particles, also known as virions, consist of two or three parts: (i) the genetic material made from either DNA or RNA, long nucleotides that carry genetic information, (ii) a protein coat, called the capsid, which surrounds and protects the genetic material, and in some cases (iii) an envelope of lipids that surrounds the protein coat when they are outside a cell. The shapes of these virus particles range from simple helical and icosahedral forms for some virus species to more complex structures for others. Thus, the term “virus”, as used herein, also encompasses viral particles.

The term “nucleocytoplasmic large DNA virus (NCLDV)”, as used herein, refers to a virus which belongs to the phylum Nucleocytoviricota. The viruses of this phylum are large, double-stranded DNA viruses with genomes ranging from 150 kb to 1.2 MB. The NCLDV infects a wide range of hosts including mammals, avian, arthropods, fish, and marine plankton. In addition, the NCLDV replicates either exclusively in the cytoplasm of the host cells or possess both cytoplasmic and nuclear stages in its life cycle. The NCLDV further comprises many genes involved in DNA repair, DNA replication, transcription, and translation. Typically, viruses with smaller genomes do not contain genes for these processes. There are nine families of NCLDVs that all share certain genomic and structural characteristics. However, it is uncertain whether the similarities of the different families of this group have a common viral ancestor. Specifically, the NCLDV phylum encompasses viruses of the Poxviridae family, an expansive viral family that includes major pathogens of humans and other mammals. Further important families in this phylum are the Asfarviridae and Iridoviridae. Viruses such as Mimiviridae that infect protists are also grouped into the Nucleocytoviricota. They can be even larger with genomes of 1200 kilobases and more than 900 genes.

The term “enveloped virus”, as used herein, refers to a virus having a viral envelope covering its protective protein capsid. The envelopes typically are derived from portions of the host cell membranes (phospholipids and proteins), but include some (viral) glycostructures such as glycoproteins and/or glycooligopeptides. Functionally, viral envelopes help viruses to enter host cells and may help them to avoid the host immune system. (Viral) glycostructures such as glycoproteins and/or glycooligopeptides on the surface of the envelopes serve to identify and bind to receptor sites on the host's membrane. The viral envelope then fuses with the host's membrane, allowing the capsid and viral genome to enter and infect the host.

Preferably, the enveloped virus is a DNA or RNA enveloped virus. More preferably, the enveloped virus is a virus of the Poxviridae family.

The Poxviridae are a diverse family with insects and vertebrates as hosts. They are large DNA viruses that replicate in the cytoplasm without a nuclear phase (a property that defines the phylum of Nucleocytoviricota and is shared with the Asfarviridae). The fully matured enveloped virions resemble flattened barrels with up to 300 nm in length and contain a genome of 128 to 365 kb that code for more than 200 genes. Poxviruses are studied as vectors for vaccination and oncotherapy due to a unique combination of virological properties. Attenuated poxviruses have been shown to be safe for recipients and able to rapidly induce a lasting and comprehensive immune response. Recombinant poxviral vectors can carry and stably maintain expression cassettes also for very large transgenes at different positions within the genome. Because poxviruses replicate without a nuclear phase risk of chromosomal aberrations is low in case of an abortive infection.

Some poxvirus species such as orf virus (genus Parapoxvirus) that naturally mainly infect sheep and goats have a narrow host range and can be further attenuated for use in humans with few genomic changes. Vaccinia virus (genus Orthopoxvirus) has a broad host range and strains such as Dairen I and Modified Vaccinia Ankara (MVA) virus were attenuated by serial passages in cell cultures that caused multiple mutations and deletions throughout the genome. The loss of certain combinations of pathogenicity factors not only reduced interference with host immunity (thus achieving attenuation) but may also have increased the beneficial immunogenic potential of some poxviral vectors.

Thus, even more preferably, the virus of the Poxviridae family is selected from the group consisting of a vaccinia virus, an avipoxvirus, and a parapoxvirus. Still even more preferably, the vaccinia virus is an attenuated vaccinia virus selected from the group consisting of a Modified Vaccinia Ankara (MVA) virus, an MVA related virus, a vaccinia virus Dairen-I, a vaccinia virus strain NYVAC, the avipoxvirus is selected from the group consisting of a canarypox virus and a fowlpox virus, or the parapoxvirus is an ORF virus.

The term “capsid”, as used herein, refers to the protein shell of a virus. It consists of several oligomeric (repeating) structural subunits made of proteins called protomers. The observable 3-dimensional morphological subunits, which may or may not correspond to individual proteins, are called capsomeres. The proteins making up the capsid of a virus are called “capsid proteins”. Alternatively, they are called “viral coat proteins (VCP)”.

The term “attenuated virus”, as used herein, refers to a virus with compromised virulence in the intended recipient, e.g. human or animal recipient. Such a property can be achieved by adaptation of a virus to narrow temperature ranges or narrow host ranges and to other artificial replication environments, including chemically defined media. Replication of such a virus is restricted in cells derived from the intended recipient, e.g. human or animal recipient, or in cells removed from the tissue environment. It may replicate to high titers outside of the intended recipient (e.g. in a permissive cell culture or laboratory animal). Specifically, Modified Vaccinia Ankara (MVA) viruses or MVA related viruses are attenuated viruses. They replicate in avian cells.

The term “highly attenuated virus”, as used herein, refers to a virus with blocked virulence in the intended recipient, e.g. human or animal recipient. Such a property can be achieved by adaptation of a virus to narrow temperature ranges or narrow host ranges and to other artificial replication environments, including chemically defined media. Replication of such a virus is blocked in cells derived from the intended recipient, e.g. human or animal recipient, or in cells removed from the tissue environment. It may replicate to high titers outside of the intended recipient (e.g. in a permissive cell/cell culture or laboratory animal). Specifically, the Modified Vaccinia Ankara (MVA) viruses or MVA related viruses can also be designated as highly attenuated viruses. They replicate in avian cells.

The term “host organism”, as used herein, refers to an organism which may be used for virus production and/or adaptation. The host organism may be a cell or an animal such as a laboratory animal. The cell may be an avian cell (e.g. a chicken, quail, goose, or duck cell such as a duck retina (CR) cell) or mammalian cell (e.g. human cell). The animal, particularly laboratory animal, may be a bird (e.g. a chicken, quail, goose, or duck), canine, mustela, rodent (e.g. a mouse, rat or hamster), an ovine, a caprine, pig, bat (e.g. a megabat or microbat) or a non-human primate (e.g. a monkey such as a great ape). Particularly, the vaccinia virus such as the MVA virus, the MVA related virus, the vaccinia virus Dairen-I, or the vaccinia virus strain NYVAC as well as the avipoxvirus such as the canarypox virus (ALVAC) or the fowlpox virus described herein replicate in an avian cell (e.g. in a chicken, quail, goose, or duck cell) or in a bird (e.g. in a chicken, quail, goose, or duck). Particularly, the parapoxvirus such as the ORF virus replicate in mammalian cells (e.g. human cells) or in a mammal (e.g. human).

The term “(host) cell”, as used herein, refers to a cell that is infected by a virus or another type of microorganism. For this virus or another type of microorganism, the host cell is a food source and/or a place to multiply or propagate.

Preferably, the (host) cell is an avian cell (e.g. in a chicken, quail, goose, or duck cell) or a mammalian cell (e.g. human cell).

The term “(host) cell line”, as used herein, refers to a permanently established cell culture that proliferates indefinitely given appropriate fresh medium and space. A cell line typically arises from a primary cell culture. Primary cultures are initiated directly from the cells, tissues, or organs of animals and are typically used in experiments within a few days. By convention, the passaging or subcultivation of a primary culture begins a cell line. However, not all primary cultures yield cell lines. Instead, the cells of some subcultivated cultures die off slowly. The adaptation of cells to grow continuously in the laboratory is a very complex process who needs a lot of experience.

The term “non-adherent (host) cell”, as used herein, refers to a cell that is able to survive in a suspension culture without being attached to a surface (e.g. tissue culture plastic carrier or micro-carrier). Said cell may be a cell which can naturally live in suspension without being attached to a surface. Said cell may also be a cell which has been modified or adapted to be able to survive in a suspension culture without being attached to a surface (e.g. tissue culture plastic carrier or micro-carrier). As mentioned above, most cells are in their original, non-modified or non-adapted form, adherent cells. A non-adherent cell can usually be grown to a higher density than adherent conditions would allow. It is, thus, more suited for culturing in an industrial scale, e.g. in a bioreactor setting or in an agitated culture, for example, in order to produce proteins such as therapeutical proteins, e.g. antibodies or hormones. To make cells attractive for the production of recombinants, the cells have to be adapted to a non-adherent cell culture. Because the original cells would undergo apoptosis under serum-free conditions and/or in the absence of a suitable surface, this adaptation is a prolonged process requiring passaging with diminishing amounts of serum (e.g. dilution rows from 10% to 0% Fetal Calve Serum (FCS)), thereby selecting an irreversibly modified cell population. Adapted non-adherent cells are known in the art. The skilled person is aware of protocols for transferring a cell from an adherent state into a non-adherent state (see, for example, Appl Microbiol Biotechnol. 2008 March; 78(3):391-9. Epub 2008 Jan. 9). Cells comprised in a (host) cell line as described herein are usually non-adherent cells.

Preferably, the (host) cell line is selected from the group consisting of an avian cell line, more preferably a chicken retina cell line AGE1.CR.PIX, a DF-1 chicken fibroblast cell line, or a duck embryonic stem cell line EB66, and a mammalian cell line, more preferably a baby hamster kidney (BHK) cell line or a human embryonal kidney 293 (HEK293) cell line.

The term “virus passaging”, as used herein, refers to a process which involves infecting a series of host organisms, e.g. cells or animals such as laboratory animals, with a virus. Each time the virus is given some time to incubate, and then the next host organism is infected with the incubated virus. This process can also be designated as “serial virus passaging”. For example, serial virus passaging allows the generation of (highly) attenuated and/or host-restricted viruses. Specifically, Modified Vaccinia Ankara (MVA) viruses or MVA related viruses are (highly) attenuated viruses. They replicate in avian cells.

When a host organism, e.g. a cell/cell line such as an avian cell/cell line or an animal such as a laboratory animal, is defined by the term “permissive”, it refers to the fact that the virus is able to circumvent defenses of said organism and is able to invade a cell, replicate in said cell, and escape from said cell. Usually this occurs when the virus has modulated one or several of the cellular intrinsic defenses of said organism and/or the immune system of said organism.

The term “host-restricted virus”, as used herein, refers to a virus which (only or mainly) replicates in a specific host organism, e.g. in a cell such as an avian cell or in an animal such as a laboratory animal. It does not replicate or only replicates at very low levels in other organisms, e.g. in other cells than avian cells. A host-restricted virus may be achieved by (serial) virus passaging of a virus in a host organism, e.g. in avian cells. Specifically, Modified Vaccinia Ankara (MVA) viruses or MVA related viruses replicate in avian cells.

The term “infectious”, as used herein, refers to the ability of a virus to replicate in a cell and to produce viral particles. Infectivity can be evaluated either by detecting the virus load or by observing disease progression in a human or in an animal.

The term “isolated enveloped virus”, as used herein, refers to a virus that is removed from its native or culturing environment. Thus, an isolated enveloped virus may be free of some or all cellular components, i.e. components of the cells in which the virus naturally occurs or in which it is cultured (e.g. cytoplasmic or membrane components). It may also be free of some or all culturing components (e.g. culture medium or culture-related impurities such as culture-remnants).

The term “purified enveloped virus”, as used herein, refers to a virus that has been isolated under conditions that reduce or eliminate the presence of unrelated materials, i.e. contaminants, including native materials, e.g. cellular debris, cellular remnants, cellular proteins, cellular DNA molecules, and/or cellular RNA molecules, from which the virus is obtained. The purified enveloped virus is preferably substantially free of cell and/or culture components. As used herein, the term “substantially free” is used operationally, in the context of analytical testing of the material. A purified enveloped virus which is substantially free of contaminants is preferably at least 50% pure, more preferably at least 90% pure, and even more preferably at least 99% or 100% pure. Purity can be evaluated by chromatography, gel electrophoresis, immunoassay, composition analysis, biological assay, and other methods known in the art.

The term “Modified Vaccinia Ankara (MVA) virus”, as used herein, refers to a highly attenuated strain of vaccinia derived from the Ankara strain and developed for use as a vaccine and vaccine adjuvant. The original MVA virus was isolated from the wild-type Ankara strain by successive passage through chicken embryonic cells. Treated thus, it lost about 15% of the genome of wild-type vaccinia including its ability to replicate efficiently in primate (including human) cells.

The MVA virus contains a single copy of a double-stranded DNA genome, approximately 178 kb in length. The viral genomic DNA comprises a core region flanked by viral telomeres. In particular, the viral telomeres are located at the left and right site of the viral genomic DNA.

Said telomeres further comprise Inverted Terminal Repeats (ITRs). The virus commonly accepted to be an MVA virus contains six characteristic deletion sites, called deletion sites I, II, III, IV, V, and VI. The numerals increase with the size of the deletion, not with position in the genome. The deletion site I is located in the left viral telomere, the deletion sites II, III, V, and VI are located in the core region. The deletion site IV is located in the right telomere. Only those viruses that contain all six deletion sites are considered bona fides MVA. The virus commonly accepted to be an MVA virus further comprises an (one) open reading frame for the (functional) gene products selected from the group consisting of C11R, C10L, D7L, A57R, B1R, B2R, B3R, B4R, B5R, B6R, B7R, B8R, B9R, B10R, B11R, B12R, B15R, B16R, B17L, B18R, B19R, and B22R. The method to describe such authentic/genuine MVAs has been published by Kremer et al., 2012. The MVA virus may have/comprise a sequence according to Genbank accession number U94848 (version U94848.1 and GI: 2772662).

The term “Modified Vaccinia Ankara (MVA) related virus”, as used herein, refers to a virus which structurally differs from the (above described or wild-type) MVA virus. It can, thus, not be considered to constitute a typical MVA virus anymore. It is rather a virus related to MVA. Said MVA related virus may, for example, not comprise a specific deletion site anymore, e.g. deletion site I, and/or comprise a specific deletion site in duplicate, e.g. deletion site IV. Alternatively or additionally, said MVA related virus may, for example, not comprise the open reading frame for a specific gene product anymore, e.g. the gene product C11, and/or comprise the open reading frame for a specific gene product in duplicate, e.g. gene product B1. A specific example of an MVA related virus is a virus in whose genome/polynucleotide the right Inverted Terminal Repeat (ITR) has been replaced the left Inverted Terminal Repeat (ITR) (Jordan et al. 2019). This rearrangement has increased the size of homology at the two termini from 15 kbp to 27 kbp. Another consequence of this rearrangement is a loss of deletion site (DS) I and a duplication of DS IV. The two DS IV loci are positioned in the center of the two ITRs: the distance of DS IV to the proximal terminus is 11 kb, and the distance to the end of the repeat and start of the genome core is 16 kb. The MVA related virus may have/comprise a sequence according to Genbank Accession number KY633487 (version KY633487.1).

The term “viral telomeres”, as used herein, refers to sequences at the left and right site of the viral genomic DNA, e.g. of the MVA virus or MVA related virus. The sequence at the left site of the viral genomic DNA is designated as left viral telomere and the sequence at the right site of the viral genomic DNA is designated as right viral telomere. The viral telomeres contain inverted terminal repeats (ITRs) that comprise or consist of regions of complementarity between the left and right sides of the genomic DNA. The viral telomeres also comprise functional and disrupted (not functional) genes that may be duplicated at both ends of the genomic DNA or that may be unique for the left or right side of the genomic DNA. Thus, viral telomeres extend beyond the mere region of the ITRs and can comprise 30 000 bp (30 kbp) or more.

The term “Inverted Terminal Repeats (ITRs)”, as used herein, refers to sequences comprising or consisting of regions of complementarity between the left and right sides of the viral genomic DNA, e.g. of the MVA virus or MVA related virus. The ITR at the left side of the genomic DNA is designated as “left ITR” and the ITR at the right side of the genomic DNA is designated as “right ITR”. The ITRs are part of the viral telomeres, in particular the left ITR is comprised in the left viral telomere and the right ITR is comprised in the right viral telomere. ITRs can form hairpin structures and appear to provide the origin for replication of the viral genomic DNA that presumably occurs by strand displacement of a leading strand and Okazaki-fragments in the lagging strand.

The term “terminus”, as used herein, refers to the left and right end of the viral genomic DNA, e.g. of the MVA virus or MVA related virus.

The term “core region”, as used herein, refers to a region of the viral genomic DNA, e.g. of the MVA virus or MVA related virus, comprising deletion sites V and III. In particular, the term “core region”, as used herein, refers to a region extending from deletion site V to deletion site III, wherein deletion sites V and III are included.

An “amino acid replacement” may also be designated herein as an “amino acid substitution”. The term “amino acid insertion”, as used herein in the context of a MVA related virus, refers to an amino acid modification which takes place within the amino acid sequence of the L3L, A3L, A34R, and/or A9L gene product(s), while the term “amino acid addition”, as used herein in the context of an MVA related virus, refers to an amino acid modification which takes place at the N- or C-terminus of the L3L, A3L, A34R, and/or A9L gene product(s).

Residues in two or more polypeptides or polynucleotides are said to “correspond” to each other if the residues occupy an analogous position in the polypeptide or polynucleotide structures. It is well known in the art that analogous positions in two or more polypeptides or polynucleotides can be determined by aligning the polypeptide or polynucleotide sequences based on amino acid sequence or nucleotide sequence similarities. Such alignment tools are well known to the person skilled in the art and can be, for example, obtained on the World Wide Web, e.g., ClustalW (www.ebi.ac.uk/clustalw) or Align (http://www.ebi.ac.uk/emboss/align/index.html) using standard settings, preferably for Align EMBOSS: needle, Matrix: Blosum62, Gap Open 10.0, Gap Extend 0.5.

The term “defensive genes” refers to cellular or viral genes that produce “defensive factors”, i.e. proteins or nucleic acids (such as interfering RNA) that interfere with viral replication (in the case of cellular defensive factors) or that neutralise the cellular defensive pathways (in the case of viral defensive factors). Many such factors are known in the literature. Examples for cellular defensive gene products are tetherins and the protein kinase R (PKR). PKR is activated by ds (double-stranded) RNA that often accompanies viral replication. If activated, PKR causes a block of translation in the infected host cells. Examples for viral defensive proteins are E3, D9 and D10 of poxviruses and g5R of African swine fever virus. The E3 protein interferes with PKR by direct interaction. However, E3 can be overwhelmed by the amount of dsRNA that accompanies replication of vaccinia viruses so that additional defensive activities are required, for example mediated by the D9 and D10 proteins. These are decapping enzymes, also known as nucleoside diphosphate hydrolases that catalyze the removal of the 7-methylguanosine cap (m⁷G) from the 5′end of mRNAs. They are proposed to preferentially decap cellular mRNAs that are required to launch and maintain an antiviral state. Decapping may furthermore promote a faster degradation also of dsRNA via the cellular Xrn1 exonuclease, and thus delay signal cascades that recognise this pathogen-associated molecular pattern. The g5R protein of ASFV is a decapping enzyme analogous to D9 and D10. The defensive genes are D9R or MVA106R for D9, and D10R or MVA107R for D10 in vaccinia viruses, and D250R for the protein g5R in ASFV. The natural decapping enzyme in human cells is the mRNA-decapping enzyme 2 encoded by the DCP2 gene.

Posttranscriptional mechanisms are important for regulation of cellular and viral gene expression. The presence of the 5′ cap structure m⁷G(5′)ppp(5′)Nm is a general feature of mRNAs that provides protection from exoribonuclease digestion and enhances translation.

Nucleocytoplasmic large DNA viruses (NCLDVs) such as vaccinia viruses and other poxviruses encode enzymes for both cap synthesis and decapping.

The term “decapping enzyme”, as used herein, refers to an enzyme that catalyzes the removal of 7-methylguanosine cap (m7G) from the 5′ end of a mRNA, producing 5′ monophosphate and releasing m7GDP(1). A mRNA decapping enzyme is capable of decapping mRNAs of various lengths and removes both Cap0 and Cap1 structures with similar efficiency. A mRNA decapping enzyme also converts 5′ triphosphate ends to 5′ monophosphate, albeit with reduced efficiency.

In addition to the key roles NCLDV such as vaccinia virus decapping enzymes play to shut off host protein synthesis and sharpen the transition between stages of the viral replication cycle, they are crucial to reduce the accumulation of viral double-stranded RNA (dsRNA) and, therefore, prevent induction of innate immune responses.

In vaccinia virus, decapping is mediated by two related enzymes, D9 and D10, which are synthesized before and after viral DNA replication, respectively. The timing of D10 synthesis correlates better with the shutdown of host gene expression, and deletion of this gene has been shown to cause persistence of host and viral mRNAs in infected cells.

The present inventors generated a new attenuated vaccinia virus, wherein the genes coding for D9 and D10 of the vaccinia virus have been replaced with the (analogous) gene coding for g5R of an African swine fever virus (ASFV). D9 and D10 as decapping enzymes in the vaccinia virus usually reduce accumulation of dsRNA, a potent trigger of innate immunity, in cells of a recipient. These decapping enzymes may even preferentially attack mRNAs important for innate immunity. The new mosaic virus that contains the gene coding for g5R in the vaccinia virus context causes a delayed but greater innate immune response which is beneficial to direct the immune system against intended targets such as tumors.

The term “tetherin”, as used herein, refers to a lipid raft associated protein. It inhibits viral infection by preventing the diffusion of virus particles after budding from infected cells. Tetherin is a coiled-coil homodimeric protein. Each monomer is equipped with two distal terminal membrane anchors, typically a transmembrane (TM) domain and a glycophospatidinylinositol (GPI) anchor. The dimers are exposed at the outer side of the plasma membrane. As viruses bud into the extracellular space, the tetherin is predicted to remain attached to the host cell at one end and to be inserted into the viral envelope at the other end. This eponymous connection prevents virions to escape and interferes with viral replication. Although tetherins are coild-coil homodimers that in principle could exist also in antiparallel configuration, deletion of one of the two membrane anchors is sufficient to disrupt the antiviral activity. Specifically, the enveloped virus of the present invention contains a polynucleotide comprising a/at least one nucleotide sequence (e.g. 1, 2, 3 or 4 nucleotide sequence(s)) encoding tetherin. Preferably, the tetherin is tetherin of chiropteran, e.g. Rousettus aegyptiacus or Desmodus retundus, human tetherin, or avian tetherin. More specifically, the enveloped virus of the present invention contains a polynucleotide comprising two nucleotide sequences encoding tetherin. The tetherins may be identical or different. Preferably, the tetherin is tetherin of chiropteran, e.g. Rousettus aegyptiacus or Desmodus retundus, human tetherin, or avian tetherin. One example tested herein is the simultaneous insertion of one tetherin into the location for thymidine kinase (TK) and one tetherin into deletion site VI. If the insertions are carried out in a poxvirus, the two insertion sites are separated by 54 kb in the poxviral genome. The two tetherins can subsequently be replaced by a gene of interest/genes of interest.

The term “essential genes” refers to viral genes that cannot be disrupted without blocking replication of a virus. Disruption can occur by removal of the promoter for such a gene or by introduction of point mutations, insertions or deletions in the coding sequence. The gene product for a disrupted essential gene must be provided in trans to restore replication. Typically, this is done with helper viruses or helper cells that contain a functional copy of the affected essential gene. Here, the essential gene of one NCLDV is replaced by the analogous essential gene of a NCLDV from a different family. A list of poxvirus essential genes can be found in Upton et al. 2003 (Upton, Slack, Hunter, Ehlers, and Roper. ‘Poxvirus Orthologous Clusters: Toward Defining the Minimum Essential Poxvirus Genome’. Journal of Virology 77, no. 13 (July 2003): 7590-7600).

The term “productive replication”, as used herein, means, in the context of virus production, that more virus can be recovered at least once from an infected culture than virus that has been added to infect the culture. The virus may cause a cytopathic effect and replicates to levels that eventually result in massive cell death in the infected culture. As opposed to productive replication, reproductive replication can occur at very low levels without accompanying cytopathic effect and may eventually lead to loss of virus in a surviving culture.

The term “disturbed replication”, as used herein, means, in the context of virus production, that a virus which differs from its wild-type/parental enveloped virus replicates at a reduced level compared to the corresponding wild-type/parental enveloped virus within a host cell/cell line. Preferably, the replication of the virus which differs from its wild-type/parental enveloped virus is at least 10-fold, 20-fold, 30-fold, 40-fold, 50-fold, 60-fold, 70-fold, 80-fold, 90-fold, 100-fold, 150-fold, 200-fold reduced compared to the corresponding wild-type/parental enveloped virus. Specifically, the enveloped virus of the present invention contains a polynucleotide comprising a/at least one nucleotide sequence encoding tetherin. As mentioned above, tetherin interferes with viral replication. Thus, when the enveloped virus of the present invention containing a polynucleotide comprising a/at least one nucleotide sequence encoding tetherin is comprised in a cell/cell line in which tetherin is expressed, said enveloped virus replicates at a reduced level compared to the corresponding wild-type/parental enveloped virus within the cell/cell line. Preferably, the replication of the enveloped virus containing a polynucleotide comprising a/at least one nucleotide sequence encoding tetherin is at least 10-fold, 20-fold, 30-fold, 40-fold, 50-fold, 60-fold, 70-fold, 80-fold, 90-fold, 100-fold, 150-fold, 200-fold reduced compared to the corresponding wild-type/parental enveloped virus. In specific cases, the enveloped virus does not reproduce itself or does not replicate at all.

The term “factor antagonizing tetherin”, as used herein, refers to a molecule which is capable of suppressing the effect of tetherin. In case a (host) cell/cell line comprises an enveloped virus containing a polynucleotide comprising a/at least one nucleotide sequence encoding tetherin and, in addition to that, expresses a factor antagonizing tetherin, the impact/effect of the tetherin expressed within the cell on the virus is diminished, preferably abolished, by the factor antagonizing tetherin.

Specifically, the replication level of the enveloped virus containing a polynucleotide comprising a/at least one nucleotide sequence encoding tetherin is increased to near a normal replication level, preferably a normal replication level, due to the presence of the factor antagonizing tetherin. In this respect, near normal replication level of the enveloped virus preferably means that the replication level of the enveloped virus is no more than 10-fold, e.g. no more than 9-fold, 8-fold, 7-fold, 6-fold, 5-fold, 4-fold, 3-fold, 2-fold, or 1-fold, below the replication level of the wild-type/parental enveloped virus of the enveloped virus. In this respect, a normal replication level of the enveloped virus preferably means that the replication level of the enveloped virus corresponds to the replication level of the wild-type/parental enveloped virus.

The factor antagonizing tetherin may exert its effect by interacting with the promoter controlling the expression of tetherin. The factor antagonizing tetherin may also act on the mRNA level by blocking tetherin mRNA. Alternatively, the factor antagonizing tetherin may perform its action by interfering with the tetherin protein.

As one example, promoter activity can be adjusted by transferring the TetR-family operator system of prokaryotes to eukaryotic cells or viruses that infect and replicate in eukaryotic cells. The TetR repressor is a dimeric protein that specifically binds to a 19 bp-long stretch of DNA. If the target sequence is proximal to a promoter then mRNA synthesis blocked. In the presence of tetracyclin (an antibiotic naturally produced by some Streptomyces bacteria) a conformational change is induced in the TetR homodimer that reduces affinity for DNA so that the repressor diffuses away from the DNA targets. With release of the transcriptional block expression of detoxifying genes is possible. In the case of the natural TetR operator the detoxifying gene is a membrane protein that selectively pumps the antibiotic out of the cell. Recombinant TetR operator sequences can also be inserted between suitable eukaryotic or viral promoters and any gene of interest to achieve regulation in ectopic systems. Often, instead of tetracyclin the synthetic substance doxycycline. is used as inducer of the deblocking change in TetR. In the present invention, a Tet Repressor (TetR) is preferably used for modulating activity of the tetherin promoter. Specifically, the Tet Repressor (TetR) modulates/is capable of modulating the activity of a Tet-Repressor (TetR) inducible promoter controlling the expression of tetherin. Particularly, the tetherin promoter is a mH52dx promoter or a HYBdx promoter, whereby dx stands for doxycycline.

Posttranscriptional regulation of expression can also be achieved with RNA interference (RNAi), a cellular process that induces nucleolytic degradation of mRNA. The involved cellular machineries are the AGO nuclease and RISC complex. The RISC complex confers specificity for certain mRNAs via small RNA segments that are homologous to the target. The small RNA segments are processed out of larger double stranded RNAs by the DICER ribonuclease. The mechanisms for control of transcription can be induced also experimentally by exogenous addition of double stranded RNA. Designed short hairpin RNAs (shRNAs) have been demonstrated to be suitable for these purposes. These shRNAs can also be expressed constitutively in the host cells for stable suppression of the targeted genes.

Preferably, the factor antagonizing tetherin is selected from the group consisting of a Tet Repressor (TetR) modulating activity of a tetherin promoter, an siRNA against tetherin, an siRNA against the mRNA encoding tetherin, a protein factor that interferes with tetherin post-translation. Specifically, the Tet Repressor (TetR) modulates/is capable of modulating the activity of a Tet-Repressor (TetR) inducible promoter controlling the expression of tetherin.

The term “nucleotide sequence of interest”, as used herein, refers to a nucleotide sequence being of interest for the skilled person and/or whose expression product is of interest for the skilled person. Preferably, the nucleotide sequence of interest is selected from the group consisting of a sequence coding for an antigen, particularly an epitope of an antigen, a diagnostic compound, and a therapeutic compound. Specifically, the nucleotide sequence of interest is a heterologous nucleotide sequence.

The term “heterologous nucleotide sequence of interest”, as used herein, refers to a nucleotide sequence that is not normally found intimately associated with the enveloped virus according to the present invention, in nature. An enveloped virus comprising a heterologous nucleotide sequence may also be designated as recombinant enveloped virus. Preferably, the heterologous nucleotide sequence of interest is selected from the group consisting of a sequence coding for an antigen, particularly an epitope of an antigen, a diagnostic compound, and a therapeutic compound.

The term “homologous recombination”, as used herein, refers to a type of genetic recombination in which genetic information is exchanged between two similar or identical molecules of double-stranded or single-stranded nucleotides (usually DNA but may also be RNA). In the context of the present invention, the/the at least one nucleotide sequence encoding tetherin is replaced by a nucleotide sequence of interest via homologous recombination.

The purity of a virus population can also be specified herein. The term “purity of a virus population”, as used herein, indicates how pure a virus population is with respect to other unwanted/undesired viruses. Said unwanted/undesired viruses may be viruses, which are not recombinant. For example, said unwanted/undesired viruses may be enveloped viruses (still) containing a polynucleotide comprising a/at least one nucleotide sequence encoding tetherin. In contrast thereto, the wanted/desired viruses may be recombinant enveloped viruses (now) containing a polynucleotide sequence comprising, instead of a nucleotide sequence encoding tetherin, a nucleotide sequence of interest. Preferably, such a virus population has a purity of at least 60%, at least 70%, at least 80%, at least 90%, at least 95%, at least 98%, at least 99% or even 100% with respect to the wanted/desired enveloped virus, e.g. the recombinant enveloped virus containing a polynucleotide sequence comprising, instead of a nucleotide sequence encoding tetherin, a nucleotide sequence of interest. Purity can be evaluated by chromatography, gel electrophoresis, immunoassay, composition analysis, biological assay, and other methods known in the art.

The term “vaccine”, as used herein, refers to an agent that can be used to elicit protective immunity in a recipient, e.g. human or animal recipient. To be effective, a vaccine can elicit immunity in a portion of the immunized population, as some individuals may fail to mount a robust or protective immune response or, in some cases, any immune response. This inability may stem from the genetic background of the recipient or because of an immunodeficiency condition (either acquired or congenital) or immunosuppression (e.g., due to treatment with chemotherapy or use of immunosuppressive drugs).

The term “epitope (also known as antigenic determinant)”, as used herein, refers to the part of an antigen that is recognized by the immune system, specifically by antibodies, B cells, or T cells.

The term “vaccination”, as used herein, means that a recipient, e.g. human or animal recipient, is challenged with an infectious virus, e.g. in an attenuated or inactivated form of said infectious virus, to induce a specific immunity.

The term “treatment”, in particular “therapeutic treatment”, as used herein, refers to any therapy which improves the health status and/or prolongs (increases) the lifespan of a subject suffering from a disease. Said therapy may eliminate the disease in a subject, arrest or slow the development of the disease in a subject, inhibit the development of the disease in a subject, decrease the severity of symptoms in a subject suffering the disease, and/or decrease the recurrence in a subject who currently has or who previously has had a disease.

The term “pharmaceutically acceptable”, as used herein, means approved by a regulatory agency of the Federal or a state government or listed in the U.S. Pharmacopeia, European Pharmacopeia (Ph. Eur.) or other generally recognized pharmacopeia for use in animals, and more particularly in humans.

The term “excipient”, as used herein, is intended to indicate all substances in a pharmaceutical composition which are not active ingredients such as binders, lubricants, thickeners, surface active agents, preservatives, emulsifiers, buffers, flavoring agents, or colorants.

The term “diluent”, as used herein, relates to a diluting and/or thinning agent. Moreover, the term “diluent” includes a solution, suspension (e.g. liquid or solid suspension) and/or media.

The term “carrier”, as used herein, relates to one or more compatible solid or liquid fillers, which are suitable for an administration, e.g. to a human. The term “carrier” relates to a natural or synthetic organic or inorganic component which is combined with an active component in order to facilitate the application of the active component, or the permeation of the active component to the intended site of action. Preferably, carrier components are sterile liquids such as water or oils, including those which are derived from mineral oil, animals, or plants, such as peanut oil, soy bean oil, sesame oil, sunflower oil, etc. Salt solutions and aqueous dextrose and glycerin solutions may also be used as aqueous carrier compounds. A carrier compound that improves or facilitates permeation of the skin in topical applications is dimethyl sulfoxide (DMSO). Another preferred carrier consists of layered double hydroxide (LDH) nanoparticles. For example, such an LDH nanoparticles can be of the form [Mg3Al(OH)8](CH3CHOHCOO), that can be obtained, for example, by a reaction of Mg(lactate)₂ 3H₂O and Al(lactate)₃ in NaOH-controlled pH at 45-65° C. and protected from CO₂ and carbonic acids. A circularized RNA may intercalate into LDH nanoparticles and can be adminstered as LDH nanoparticle or be co-administered with VLPs.

Pharmaceutical carriers, diluents, and/or excipients can be selected with regard to the intended route of administration and standard pharmaceutical practice. The pharmaceutical compositions of the present invention may comprise as, or in addition to, the carrier(s), excipient(s) or diluent(s) any suitable binder(s), lubricant(s), suspending agent(s), coating agent(s), and/or solubilising agent(s). Examples of suitable binders include starch, gelatin, natural sugars such as glucose, lactose, sucrose, trehalose, corn sweeteners, natural and synthetic gums, such as acacia, tragacanth or sodium alginate, carboxymethyl cellulose, and polyethylene glycol. Examples of suitable lubricants include sodium oleate, sodium stearate, magnesium stearate, sodium benzoate, sodium acetate, sodium chloride, and the like. Preservatives, stabilizers, dyes, and even flavoring agents may be provided in the pharmaceutical composition. Examples of preservatives include sodium benzoate, sorbic acid, and esters of p-hydroxybenzoic acid. Antioxidants and suspending agents may be also used.

The term “subject”, as used herein, refers to any individual which may receive the virus as described herein. The term “subject”, as used herein, refers to any individual that/who may benefit from the treatment with the virus as described herein.

The subject may be a vertebrate, e.g. a human being, dog, cat, sheep, goat, cow, horse, camel or pig. It is particularly preferred that the “subject” is a human being.

The terms “subject”, “individual”, or “patient” are used interchangeably herein.

### EMBODIMENTS OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

As mentioned above, for homologous recombination, one challenge is that very few recombinant viruses are generated and that these are difficult to identify in the adherent cell culture monolayers. The few recombinants must be identified, marked and isolated (picked) by experienced operators with pipettes that directly access the infected monolayer. The isolated plaques are resuspended in medium and transferred to a fresh monolayer. Again, usually after 48-72 h, recombinant plaques must be identified, isolated and again transferred. This process is repeated until parental virus has been completely removed. Plaque picking is associated with a risk for contamination because of the additional manipulation (pipet tips, agarose overlay, animal sera in the adherent culture medium) and from internal sources (neighboring plaques with parental virus). The fitness of recombinant viruses can be lower compared to the parental virus depending on the desired transgenes that are being expressed. This provides a replication advantage to the parental virus. Depending on the fitness of the recombinant virus between 5-8 cycles of plaque purification are required until a pure recombinant preparation is obtained.

For rescue of recombinant viruses out of bacterial artificial chromosomes (BACs) the challenges start with manipulation of the very large infectious DNA constructs. Recombination is assisted by induced marker deletion in special strains of E. coli bacteria. The BACs are purified and transfected into suitable host cells. After infection with the helper virus the recombinant progeny is formed out of the BACs, catalyzed by the replication machinery that is provided in trans by the helper virus.

Again, the challenge is to identify the desired recombinants. Identification can be facilitated by insertion of a fluorescent marker into the viruses in such a way, that the marker is framed by smaller homologous flanks (Kugler et al. 2019). The marker is deleted by internal recombination via sequential passaging until a pure and marker-free preparation is achieved. The helper virus can be a related poxvirus such as fowlpox virus or rabbit fibroma virus. If a helper virus is used that can also replicate in avian cells then separation from the desired recombinants is an additional challenge. If a helper virus is used with impaired replication in avian cells then an additional cellular substrate is required specifically for production of the helper virus. In both cases, helper viruses that are not part of the final composition need to be prepared at sufficient purity and documentation which, together with dependence on animal sera for cultivation, complicate industrial application especially under GMP regimes.

The present invention facilitates the derivation of recombinant large nucleocytoplasmic DNA viruses, including poxviruses, drastically. The present inventors developed a process that allows to obtain the desired recombinants free of contamination with parental virus within a single passage. Only the parental virus and a shuttle plasmid comprising the gene of interest are required. The system is suitable for rescue of recombinant viruses either in adherent cells or in suspension cells that proliferate in chemically defined medium.

The principle of the present invention is to equip the receiving virus with a factor that impairs the ability of the parental virus to counter innate immunity cascades of a host cell or that is derived from the innate immune system of a host cell. The immune factor occupies at least one of the intended insertion sites and is highly efficient in interfering with replication of the receiving virus. Recombination with a gene of interest removes the immune factor and allows replication only of desired recombinants. Here, the present inventors used tetherin as one example for attenuation of replication. The gene of interest can be a fluorescent marker for quantification of efficiency or any therapeutically active gene such as vaccine antigen, immune enhancer or protein for molecular replacement.

To obtain a parental virus that is highly impaired as described here, the immune factor needs to be suppressed during replication. Here, the present inventors used, as one example, a Tet-Repressor (TetR) inducible promoter in the parental virus that is silent under normal conditions in the fully permissive host cell. The TetR is constitutively expressed by the host cell and can be deactivated by addition of doxycycline. Other inducible and non-inducible systems (such as RNA interference) are conceivable. The host cell can be of a lineage that proliferates in the adherent phase in presence of animal serum, but it can also be of a lineage that proliferates in suspension in chemically defined medium for industrial processes under GMP regimes. Using the TetR system has the advantage that only a single cell line is required for production of receiving virus, generation of recombinant viruses and production of the final recombinant viruses. Addition of doxycycline is required only once, for one passage at any time after the recombination.

The above described new approach is not time-consuming, inexpensive, simple, and does not promote contamination.

Thus, in a first aspect, the present invention relates to an (a synthetic) enveloped virus containing a polynucleotide comprising a/at least one nucleotide sequence (e.g. 1, 2, 3, or 4 nucleotide sequence(s)) encoding tetherin.

The polynucleotide contained within the virus may represent the viral genome.

The replication of such an enveloped virus, if present in a (non-modified) host cell, is disturbed. An enveloped virus whose replication is disturbed replicates at a reduced level compared to the corresponding wild-type/parental enveloped virus. Preferably, the replication of the enveloped virus containing a polynucleotide comprising a/at least one nucleotide sequence encoding tetherin is at least 10-fold, 20-fold, 30-fold, 40-fold, 50-fold, 60-fold, 70-fold, 80-fold, 90-fold, 100-fold, 150-fold, 200-fold reduced compared to the corresponding wild-type/parental enveloped virus. In specific cases, the enveloped virus does not reproduce itself or does not replicate at all.

Specifically, the enveloped virus is a virus whose replication is affected by (heterologous) tetherin. Tetherin is a lipid raft associated protein. It inhibits enveloped virus infection by preventing the diffusion of virus particles after budding from infected cells. Thus, the presence of tetherin in a (non-modified) host cell, expressed from the/the at least one nucleotide sequence encoding tetherin comprised in the polynucleotide of the virus, leads to the disturbance of the replication of the virus in the host cell. Particularly, an enveloped virus whose replication is disturbed replicates at a reduced level compared to the corresponding wild-type/parental enveloped virus. Preferably, the replication of the enveloped virus containing a polynucleotide comprising a/at least one nucleotide sequence encoding tetherin is at least 10-fold, at least 20-fold, at least 30-fold, at least 40-fold, at least 50-fold, at least 60-fold, at least 70-fold, at least 80-fold, at least 90-fold, at least 100-fold, at least 150-fold, or at least 200-fold reduced compared to the corresponding wild-type/parental enveloped virus. In specific cases, the enveloped virus does not reproduce itself or does not replicate at all.

The skilled person knows how to measure the viral replication efficiency or the infectious yield of the synthetic enveloped virus and the wild-type/parental enveloped virus and to compare the different parameters with each other. The skilled person also knows how to determine the replication kinetics in order to compare the performance of the synthetic enveloped virus with the wild-type/parental enveloped virus.

The enveloped virus may be a DNA or RNA enveloped virus. Preferably, the enveloped virus is a virus of the Poxviridae family. More preferably, the Poxviridae family is selected from the group consisting of a vaccinia virus, an avipoxvirus, and a parapoxvirus. Even more preferably,
the vaccinia virus is an attenuated vaccinia virus selected from the group consisting of a Modified Vaccinia Ankara (MVA) virus, an MVA related virus, a vaccinia virus Dairen-I, a vaccinia virus strain NYVAC,
the avipoxvirus is selected from the group consisting of a canarypox virus and a fowlpox virus, or
the parapoxvirus is an ORF virus.

Thus, in one preferred embodiment, the present invention relates to a (synthetic) vaccinia virus, particularly Modified Vaccinia Ankara (MVA) virus, MVA related virus, vaccinia virus Dairen-I, or vaccinia virus NYVAC, containing a polynucleotide comprising a/at least one nucleotide sequence encoding tetherin. In one another preferred embodiment, the present invention relates to a (synthetic) avipoxvirus, particularly canarypox virus or fowlpox virus, containing a polynucleotide comprising a/at least one nucleotide sequence encoding tetherin. In one another preferred embodiment, the present invention relates to a (synthetic) parapoxvirus, particularly ORF virus, containing a polynucleotide comprising a/at least one nucleotide sequence encoding tetherin.

The/the at least one nucleotide sequence encoding tetherin is comprised in the polynucleotide of the virus at a position suitable for the incorporation of a nucleotide sequence of interest. The suitable position may be a natural deletion site or an intergenic site.

In case of an MVA virus, this position is preferably selected from the group consisting of deletion site I, deletion site II, deletion site III, deletion site IV, deletion site V, deletion site VI, between two essential or non-essential genes or replacement of one or more non-essential gene(s) including the tyrosine kinase gene.

In case of an MVA related virus, this position is preferably selected from the group consisting of deletion site II, deletion site III, deletion size IV, deletion site V, deletion size IV, between two essential or non-essential genes or replacement of one or more non-essential gene(s) including the tyrosine kinase gene.

The tetherin may be any tetherin. Specifically, heterologous tetherin. Preferably, the tetherin is tetherin of a chiropteran, more preferably Rousettus aegyptiacus or Desmodus retundus, human tetherin, or avian tetherin.

As mentioned above, the enveloped virus can comprise a/at least one nucleotide sequence (e.g. 1, 2, 3, 4 nucleotide sequence(s)) encoding tetherin. The tetherins (if more than one) are always at different positions within the virus genome. For example, the tetherins are comprised in deletion site I, deletion site II, deletion site III, deletion site IV, deletion site V, deletion site VI, between two essential or non-essential genes or replace one or more non-essential gene(s) including the tyrosine kinase gene.

Specifically, the enveloped virus of the present invention contains a polynucleotide comprising one nucleotide sequence encoding tetherin. Preferably, the tetherin is tetherin of chiropteran such as Rousettus aegyptiacus or Desmodus retundus. More preferably, the tetherin is comprised in deletion site I, deletion site II, deletion site III, deletion site IV, deletion site V, deletion site VI, between two essential or non-essential genes or replaces of one or more non-essential gene(s) including the tyrosine kinase gene. The insertion of chiropteran tetherin into the location for thymidine kinase (TK) or into deletion site VI is particularly preferred.

More specifically, the enveloped virus of the present invention contains a polynucleotide comprising two nucleotide sequences encoding tetherin. The tetherins may be identical or different. Preferably, the tetherin is tetherin of chiropteran such as Rousettus aegyptiacus or Desmodus retundus. The tetherins are always at different positions within the virus genome. More preferably, the tetherins are comprised in deletion site I, deletion site II, deletion site III, deletion site IV, deletion site V, deletion site VI, between two essential or non-essential genes or replace one or more non-essential gene(s) including the tyrosine kinase gene. The insertion of chiropteran tetherin into the location for thymidine kinase (TK) and into deletion site VI is particularly preferred.

To ensure that tetherin is expressed in a host cell comprising the enveloped virus of the present invention, the/the at least one nucleotide sequence encoding tetherin is under control of/operably linked to a promoter. Preferably, the promoter is selected from the group consisting of an early promoter, a late promoter, more preferably P11 promoter, an intermediate promoter, or a synthetic early/late promoter, more preferably a Tet-Repressor (TetR) inducible promoter such as a mH52dx (dx=doxycycline responsive by insertion of one or several Tet operator (tetO) element(s)) promoter or a HYBdx (dx=doxycycline responsive by insertion of one or several tetO element(s)) promoter.

Thus, in one more preferred embodiment, the present invention relates to a (synthetic) vaccinia virus, particularly Modified Vaccinia Ankara (MVA) virus, MVA related virus, vaccinia virus Dairen-I, or vaccinia virus NYVAC, containing a polynucleotide comprising a/at least one nucleotide sequence encoding tetherin, wherein tetherin is under control of a P11 promoter or a Tet-Repressor (TetR) inducible promoter such as a mH52dx promoter or a HYBdx promoter. In one another more preferred embodiment, the present invention relates to a (synthetic) avipoxvirus, particularly canarypox virus or fowlpox virus, containing a polynucleotide comprising a/at least one nucleotide sequence encoding tetherin, wherein tetherin is under control of a P11 promoter or a Tet-Repressor (TetR) inducible promoter such as a mH52dx promoter or a HYBdx promoter. In one another more preferred embodiment, the present invention relates to a (synthetic) parapoxvirus, particularly ORF virus, containing a polynucleotide comprising a/at least one nucleotide sequence encoding tetherin, wherein tetherin is under control of a P11 promoter or a Tet-Repressor (TetR) inducible promoter such as a mH52dx promoter or a HYBdx promoter.

In one even more preferred embodiment, the present invention relates to a (synthetic) Modified Vaccinia Ankara (MVA) virus or an (a synthetic) MVA related virus containing a polynucleotide comprising a/at least one nucleotide sequence encoding tetherin,
wherein the/the at least one nucleotide sequence encoding tetherin is comprised in deletion site I, deletion site II, deletion site III, deletion site IV, deletion site V, deletion site VI, between two essential or non-essential genes or replaces one or more non-essential gene(s) including the tyrosine kinase gene of said virus, and/or
wherein the/the at least one nucleotide sequence encoding tetherin is under control of a P11 promoter or a Tet-Repressor (TetR) inducible promoter such as a mH52dx promoter or a HYBdx promoter.

In one still even more preferred embodiment, the present invention relates to a (synthetic) Modified Vaccinia Ankara (MVA) virus or an (a synthetic) MVA related virus containing a polynucleotide comprising a/at least one nucleotide sequence encoding tetherin, wherein the/the at least one nucleotide sequence encoding tetherin is comprised in deletion site I, deletion site II, deletion site III, deletion site IV, deletion site V, deletion site VI, between two essential or non-essential genes or replaces one or more non-essential gene(s) including the tyrosine kinase gene of said virus,
wherein the/the at least one nucleotide sequence encoding tetherin is under control of a P11 promoter or a Tet-Repressor (TetR) inducible promoter such as a mH52dx promoter or a HYBdx promoter, and/or
wherein the tetherin is tetherin of chiropteran, particularly Rousettus aegyptiacus or Desmodus retundus.

Particularly, the Modified Vaccinia Ankara (MVA) related virus, as described herein, comprises (differs from (wild-type) MVA by) one or more, e.g. 1, 2, 3, 4, 5, 6, 7, or 8, of the following features:
(i) a nucleotide sequence corresponding to a region that includes the right Inverted Terminal Repeat (ITR) and extends to but excludes deletion site III instead of (replacing) a nucleotide sequence that includes the left Inverted Terminal Repeat (ITR) and extends to but excludes deletion site V,
(ii) two copies of a nucleotide sequence comprising deletion site IV and the right ITR, (iii) no nucleotide sequence comprising deletion site I and the left ITR,
(iv) no deletion site I,
(v) two deletion sites IV,
(vi) no open reading frame for at least one gene product selected from the group consisting of C11R, C10L, and D7L,
(vii) two open reading frames for at least one gene product selected from the group consisting of A57R, B1R, B2R, B3R, B4R, B5R, B6R, B7R, B8R, B9R, B10R, B11R, B12R, B15R, B16R, B17L, B18R, B19R, and B22R, and/or
(viii) a nucleotide sequence encoding a L3L gene product, wherein said nucleotide sequence comprises at least one mutation resulting in an amino acid sequence modification of said gene product.

The skilled person will be understood that the nucleotide sequences mentioned above are part of/comprised in the viral genomic DNA of the MVA related virus or are part of/comprised in the genome of the MVA related virus.

More particularly,
(i) the region that includes the right ITR and extends to but excludes deletion site III has a nucleotide sequence according Genbank Accession number KY633487 (preferably ranging from nucleotide position 162221 to 190549 or a nucleotide position corresponding thereto) or is a variant thereof which is at least 85%, at least 90%, at least 95% or 100%, e.g. at least 85, 86, 87, 88, 89, 90, 91, 92, 93, 94, 95, 96, 97, 98, 99% or 100%, identical to said nucleotide sequence,
(ii) the region that includes the left ITR and extends to but excludes deletion site V has a nucleotide sequence according to Genbank Accession number KY633487 (preferably ranging from nucleotide position 1 to 31261 or a nucleotide position corresponding thereto) or is a variant thereof which is at least 85%, at least 90%, at least 95% or 100%, e.g. at least 85, 86, 87, 88, 89, 90, 91, 92, 93, 94, 95, 96, 97, 98, 99% or 100%, identical to said nucleotide sequence, and/or
(iii) the nucleotide sequence comprising deletion site IV and the right ITR has a nucleotide sequence according to Genbank Accession number KY633487 (preferably ranging from nucleotide position 179272 to 190549 or a nucleotide position corresponding thereto) or is a variant thereof which is at least 85%, at least 90%, at least 95% or 100%, e.g. at least 85, 86, 87, 88, 89, 90, 91, 92, 93, 94, 95, 96, 97, 98, 99% or 100%, identical to said nucleotide sequence.

Even more particularly, the MVA related virus (further) comprises a nucleotide sequence encoding a L3L gene product, an A3L gene product, an A34R gene product and an A9L gene product, wherein said nucleotide sequence comprise at least one mutation (e.g. 1, 2, 3, or 4 mutation(s)) resulting in an/at least one amino acid sequence modification (e.g. 1, 2, 3, or 4 amino acid modification(s)) of said gene products (i.e. said L3L gene product, said A3L gene product, said A34R gene product and said A9L gene product).

As to the MVA related virus and its modifications compared to the MVA virus, it is referred to WO 2018/153460 A1, specifically to pages 15 to 24 of the description of WO 2018/153460 A1. These pages are incorporated herein by reference.

To obtain an enveloped virus that is highly impaired as described above, the immune factor tetherin needs to be suppressed during replication.

Thus, in a second aspect, the present invention relates to a cell line expressing a factor antagonizing tetherin. Said cell line is particularly a cell line which grows in suspension culture.

Especially, the factor antagonizing tetherin is expressed from a polynucleotide stably maintained in said cell line.

Preferably, the cell line is permissive for the enveloped virus which represents the corresponding wild-type/parental enveloped virus of the virus of the first aspect. More preferably, the virus of the first aspect is capable of replicating in the cell line of the second aspect.

Specifically, the factor antagonizing tetherin expressed in the cell line is capable of suppressing the effect of tetherin expressed by the virus of the first aspect. Thus, in a cell line which comprises an enveloped virus containing a polynucleotide comprising a/at least one nucleotide sequence encoding tetherin and, in addition to that, expresses a factor antagonizing tetherin, the impact/effect of the tetherin expressed within the cell line on the virus is diminished, preferably abolished, by the factor antagonizing tetherin.

More specifically, the replication level of the enveloped virus containing a polynucleotide comprising a/at least one nucleotide sequence encoding tetherin is increased to near a normal replication level, preferably a normal replication level, due to the presence of the factor antagonizing tetherin. In this respect, near normal replication level of the enveloped virus preferably means that the replication level of the enveloped virus is no more than 10-fold, e.g. no more than 9-fold, 8-fold, 7-fold, 6-fold, 5-fold, 4-fold, 3-fold, 2-fold, or 1-fold, below the replication level of the wild-type/parental enveloped virus of the enveloped virus. In this respect, a normal replication level of the enveloped virus preferably means that the replication level of the enveloped virus corresponds to the replication level of the wild-type/parental enveloped virus.

The factor antagonizing tetherin may exert its effect by interacting with the promoter controlling the expression of tetherin. The factor antagonizing tetherin may also act on the mRNA level by blocking tetherin mRNA. Alternatively, the factor antagonizing tetherin may perform its action by interfering with the tetherin protein.

In one preferred embodiment, the factor antagonizing tetherin is selected from the group consisting of a Tet Repressor (TetR) which modulates/is capable of modulating the activity of a tetherin promoter, an siRNA against tetherin, an siRNA against the mRNA encoding tetherin, and a protein factor that interferes with tetherin post-translation. Specifically, the Tet Repressor modulates/is capable of modulating the activity of a Tet-Repressor (TetR) inducible promoter controlling the expression of tetherin.

Particularly, the Tet Repressor (TetR) reduces, especially abolishes, the activity of the promoter which controls tetherin expression in the enveloped virus.

Particularly, the siRNA is directed against an element in the mRNA for tetherin.

Particularly, the protein factor that interferes with tetherin post-translation is nef or vpu.

Herein, the present inventors used, as one example, a Tet-repressor (tetR) inducible promoter to control expression of tetherin. This promoter is silent under normal conditions in a fully permissive host cell. The TetR is constitutively expressed by the host cell of the second aspect and, thus, allows propagation of the enveloped virus containing a polynucleotide comprising a/at least one nucleotide sequence encoding tetherin. It can be deactivated by the addition of doxycycline. Other inducible and non-inducible systems (such as RNA interference) are possible. The host cell can be of a lineage that proliferates in the adherent phase in presence of animal serum, but it can also be of a lineage that proliferates in suspension in chemically defined medium for industrial processes under GMP regimes. Using the TetR system has the advantage that only a single cell line is required for the production of receiving virus, generation of recombinant viruses and production of the final recombinant viruses. The addition of doxycycline is required only once, for one passage at any time after the recombination.

In one more preferred embodiment, the cell line is selected from the group consisting of an avian cell line, particularly a chicken retina cell line AGE1.CR.PIX, a DF-1 chicken fibroblast cell line, or a duck embryonic stem cell line EB66, and a mammalian cell line, particularly a baby hamster kidney (BHK) cell line or a human embryonal kidney 293 (HEK293) cell line.

In one even more preferred embodiment,
the cell line expressing a factor antagonizing tetherin is an avian cell line, particularly a chicken retina cell line AGE1.CR.PIX, a DF-1 chicken fibroblast cell line, or a duck embryonic stem cell line EB66, and
the factor antagonizing tetherin is a Tet Repressor (TetR) which modulates/is capable of modulating the activity of a Tet-Repressor (TetR) inducible promoter controlling the expression of tetherin.

Alternatively,
the cell line expressing a factor antagonizing tetherin is a mammalian cell line, particularly a baby hamster kidney (BHK) cell line or a human embryonal kidney 293 (HEK293) cell line, and
the factor antagonizing tetherin is a Tet Repressor (TetR) which modulates/is capable of modulating the activity of a Tet-Repressor (TetR) inducible promoter controlling the expression of tetherin.

In one still even more preferred embodiment,
the cell line expressing a factor antagonizing tetherin is an avian cell line, particularly a chicken retina cell line AGE1.CR.PIX, a DF-1 chicken fibroblast cell line, or a duck embryonic stem cell line EB66,
the factor antagonizing tetherin is a Tet Repressor (TetR) which modulates/is capable of modulating the activity of a Tet-Repressor (TetR) inducible promoter controlling the expression of tetherin, and
tetherin is tetherin of chiropteran, particularly Rousettus aegyptiacus or Desmodus retundus.

Alternatively,
the cell line expressing a factor antagonizing tetherin is a mammalian cell line, particularly a baby hamster kidney (BHK) cell line or a human embryonal kidney 293 (HEK293) cell line, the factor antagonizing tetherin is a Tet Repressor (TetR) which modulates/is capable of modulating the activity of a Tet-Repressor (TetR) inducible promoter controlling the expression of tetherin, and
tetherin is tetherin of chiropteran, particularly Rousettus aegyptiacus or Desmodus retundus.

In a third aspect, the prevent invention relates to a method for propagating an enveloped virus containing a polynucleotide comprising a/at least one nucleotide sequence encoding tetherin comprising the step of:
culturing the enveloped virus containing a polynucleotide comprising a/at least one nucleotide sequence encoding tetherin in a cell line expressing a factor antagonizing tetherin.

Specifically, the cell line has been (before/previously)
infected with the enveloped virus containing a polynucleotide comprising a/at least one nucleotide sequence encoding tetherin, or
transfected with one or more nucleotide sequence(s) encoding said enveloped virus.

As to the preferred embodiments of the enveloped virus, it is referred to the enveloped virus as defined in the first aspect. Preferably, the enveloped virus is a virus according to the first aspect.

As to the preferred embodiments of the cell line, it is referred to the cell line as defined in the second aspect. Preferably, the cell line is a cell line according to the second aspect.

More preferably, the enveloped virus is a virus according to the first aspect and the cell line is a cell line according to the second aspect.

Culturing may be performed according to standard procedures readily available to the skilled person. In this respect, is should be noted that culture/incubation times are important for the production of the enveloped virus in high yields. In addition, since the integral of viable cell density (IVCD) is one of the factors that impact the overall volumetric productivity in cell culture, it is desirable to keep cell viability at a high level over the duration of upstream processing. Further, nutrient depletion, extended exposure to impeller shear forces and other factors that occur during culturing may constitute apoptotic stimuli which serve to decrease cell viability and which should, thus, be avoided.

The culturing time may range from between 1 day and 24 days, e.g. 1, 2, 3, 4, 5, 6, 7, 8, 9, 10, 11, 12, 13, 14, 15, 16, 17, 18, 19, 20, 21, 22, 23, or 24 day(s).

The enveloped virus culture may be an agitated culture or a culture in a bioreactor. Bioreactors are generally categorized similarly to chemical reactors according to their mixing characteristics. Said bioreactor may be a (well mixed) stirred tank reactor or a plug flow (tubular) reactor. In an ideal well-mixed bioreactor, the mixing is assumed to be intense enough that the fluid (cells and culture medium) is homogenous through the reactor. The bioreactor may be a fed-batch, batch, or continuous bioreactor or the culturing process may be a batch, fed-batch or continuous culturing process.

The method may further comprise the step of isolating/harvesting the propagated enveloped virus containing a polynucleotide comprising a/at least one nucleotide sequence encoding tetherin from said cell line.

Various isolation/harvesting procedures are known in the art for enveloped viruses which escape their host cells after their production. For example, the isolation/harvesting is achieved by separating the enveloped viruses from the cells via centrifugation, sedimentation and/or filtration, e.g. via centrifugation and filtration, via sedimentation and filtration, via sedimentation and centrifugation, or via centrifugation, sedimentation, and filtration. Depending on the enveloped virus to be harvested, the parameters for centrifugation, sedimentation or filtration may vary. The person skilled in the art is able to easily adapt the appropriate separation parameters, e.g. the acceleration-force/G-force and/or time using centrifugation for separation, filter size using filtration for separation, and/or sedimentation time using sedimentation for separation, in order to harvest the enveloped viruses produced by said cells.

In one even more preferred embodiment,
the cell line expressing a factor antagonizing tetherin is an avian cell line, particularly a chicken retina cell line AGE1.CR.PIX, a DF-1 chicken fibroblast cell line, or a duck embryonic stem cell line EB66,
the factor antagonizing tetherin is a Tet Repressor (TetR) which modulates/is capable of modulating the activity of a Tet-Repressor (TetR) inducible promoter controlling the expression of tetherin, and
the enveloped virus containing a polynucleotide comprising a/at least one nucleotide sequence encoding tetherin is a vaccinia virus, such as a MVA virus, an MVA related virus, a vaccinia virus Dairen-I, or a vaccinia virus NYVAC, or a avipoxvirus, such as a canarypox virus (ALVAC) or a fowlpox virus.

Alternatively,
the cell line expressing a factor antagonizing tetherin is a mammalian cell line, particularly a baby hamster kidney (BHK) cell line or a human embryonal kidney 293 (HEK293) cell line,
the factor antagonizing tetherin is a Tet Repressor (TetR) which modulates/is capable of modulating the activity of a Tet-Repressor (TetR) inducible promoter controlling the expression of tetherin, and
the enveloped virus containing a polynucleotide comprising a/at least one nucleotide sequence encoding tetherin is a parapoxvirus, such as an ORF virus.

In the above embodiment, the tetherin is specifically tetherin of chiropteran, particularly Rousettus aegyptiacus or Desmodus retundus.

In one still even more preferred embodiment,
the cell line expressing a factor antagonizing tetherin is an avian cell line, particularly a chicken retina cell line AGE1.CR.PIX, a DF-1 chicken fibroblast cell line, or a duck embryonic stem cell line EB66,
the factor antagonizing tetherin is a Tet Repressor (TetR), and
the enveloped virus containing a polynucleotide comprising a/at least one nucleotide sequence encoding tetherin is a Modified Vaccinia Ankara (MVA) virus or an MVA related virus containing a polynucleotide comprising a/at least one nucleotide sequence encoding tetherin, wherein the/the at least one nucleotide sequence encoding tetherin is comprised in deletion site I, deletion site II, deletion site III, deletion site IV, deletion site V, deletion site VI, or tyrosine kinase deletion site of said virus, and/or
wherein the/the at least one nucleotide sequence encoding tetherin is under control of a Tet-Repressor (TetR) inducible promoter such as a mH52dx promoter or a HYBdx promoter.

In the above embodiment, the tetherin is specifically tetherin of chiropteran, particularly Rousettus aegyptiacus or Desmodus retundus.

The principle of the present invention is to equip the receiving virus with tetherin which is a factor that impairs the ability of the parental virus to counter innate immunity cascades of a host cell. The immune factor tetherin is highly efficient in interfering with replication of the receiving virus as described herein. Recombination with a gene/nucleotide sequence of interest removes the immune factor tetherin and allows replication only of desired recombinants. Viruses that have not recombined cannot replicate to levels that compete with the desired recombined viruses. This approach simplifies the selection of recombinants. Only one infection virus passage is required to achieve recombinant viruses of sufficient purity. The gene/nucleotide sequence of interest can be a fluorescent marker for quantification of efficiency or any therapeutically active gene such as vaccine antigen, immune enhancer or protein for molecular replacement. If two or more nucleotide sequences encoding a tetherin are comprised in the virus, two or more nucleotide sequences encoding tetherin can be replaced by a gene/nucleotide sequence of interest. The replaced gene/nucleotide sequence of interest can be the same or different, i.e. in one case, the same gene/nucleotide sequence of interest can be placed in the place of the nucleotide sequence encoding tetherin and in one another case, different genes/nucleotide sequences of interest can be placed in the place of the nucleotide sequence encoding tetherin.

Thus, in a fourth aspect, the present invention relates to method for producing a virus population comprising a recombinant enveloped virus (containing a polynucleotide comprising a nucleotide sequence of interest) comprising the steps of:
(i) transfecting a cell line which
   (a) expresses a factor antagonizing tetherin and
   (b) comprises an enveloped virus containing a first polynucleotide comprising a/at least one nucleotide sequence encoding tetherin
   with a second polynucleotide comprising a nucleotide sequence of interest,
   wherein the second polynucleotide is capable of homologous recombination with the first polynucleotide contained in the enveloped virus, and
(ii) culturing the enveloped virus in the cell line transfected in (i), thereby obtaining a virus population that is composed of a recombinant enveloped virus comprising, instead of the/the at least one nucleotide sequence encoding tetherin, the nucleotide sequence of interest, and an enveloped virus containing the first polynucleotide (comprising a/at least one nucleotide sequence encoding tetherin).

The second polynucleotide comprising a nucleotide sequence of interest preferably represents a shuttle plasmid.

As to the preferred embodiments of the enveloped virus, it is referred to the enveloped virus as defined in the first aspect. Preferably, the enveloped virus is a virus according to the first aspect.

As to the preferred embodiments of the cell line, it is referred to the cell line as defined in the second aspect. Preferably, the cell line is a cell line according to the second aspect.

More preferably, the enveloped virus is a virus according to the first aspect and the cell line is a cell line according to the second aspect.

Specifically, the cell line transfected in step (i) has been infected with an enveloped virus containing a first polynucleotide comprising a/at least one nucleotide sequence encoding tetherin before step (i).

In one preferred embodiment, the method further comprises the steps of:
(iii) infecting a cell line (not expressing a factor antagonizing tetherin) with the virus population of step (ii), and
(iv) culturing the virus population, thereby
   separating the recombinant virus comprising, instead of the/the at least one nucleotide sequence encoding tetherin, the nucleotide sequence of interest, from the enveloped virus containing the first polynucleotide (comprising a/at least one nucleotide sequence encoding tetherin), or
   enriching the recombinant virus comprising, instead of the/the at least one nucleotide sequence encoding tetherin, the nucleotide sequence of interest.

The cell line not expressing a factor antagonizing tetherin may be a cell line which is naïve with respect to a factor antagonizing tetherin. Thus, it may be any cell line not permissive for tetherin. The cell line not expressing a factor antagonizing tetherin may, however, also be a cell line in which the effect of the factor antagonizing tetherin is blocked/suppressed/switched off. In this respect, a cell line in which the factor antagonizing tetherin is a Tet Repressor (TetR) which is able to suppress the activity of a Tet Repressor (TetR) inducible promoter, e.g. a mH52dx promoter or a HYBdx promoter, controlling the expression of tetherin in the enveloped virus is preferred. The TetR is constitutively expressed by the host cell and can be deactivated by addition of doxycycline (before/in step (iii)). Using the constitutively active TetR system has the advantage that only a single cell line is required for the production of the receiving virus (see method of the third aspect), for the generation of recombinant viruses (see step (ii) of the method of the fourth aspect) and for the production of the final recombinant viruses (see step (iv) of the method of the fourth aspect). The addition of doxycycline is required only once, for one passage at any time after the homologous recombination event.

If desired, the above-mentioned steps (iii) and (iv) may be repeated one or more times (e.g. 1, 2, or 3 times) to further separate or enrich the recombinant virus in the virus population. However, as shown in the experimental section, one passage is already sufficient to purify the recombinant virus to a purity of about 95% or more.

In one more preferred embodiment, the method further comprises the steps of:
(v) isolating the virus population from the cell line, wherein the virus population is mainly composed, preferably composed, of a recombinant enveloped virus comprising, instead of the/the at least one nucleotide sequence encoding tetherin, the nucleotide sequence of interest.

The purity of a virus population finally indicates how pure the virus population is with respect to unwanted/undesired non-recombinant viruses. Said unwanted/undesired non-recombinant viruses are enveloped viruses (still) containing a polynucleotide comprising a/at least one nucleotide sequence encoding tetherin. In contrast thereto, the wanted/desired recombinant viruses are the recombinant enveloped viruses (now) containing a polynucleotide sequence comprising, instead of a/at least one nucleotide sequence encoding tetherin, a nucleotide sequence of interest. Preferably, such a virus population has a purity of at least 60% or at least 70% with respect to the wanted/desired recombinant enveloped virus. More preferably, such a virus population has a purity of at least 80% or at least 90% with respect to the wanted/desired recombinant enveloped virus. Even more preferably, such as virus population has a purity of at least 95%, at least 98%, or at least 99% with respect to the wanted/desired recombinant enveloped virus. Still even more preferably, the virus population has a purity of 100% with respect to the wanted/desired recombinant enveloped virus, e.g. the recombinant enveloped virus containing a polynucleotide sequence comprising, instead of a/at least one nucleotide sequence encoding tetherin, a nucleotide sequence of interest.

If two or more nucleotide sequences encoding a tetherin are comprised in the virus, two or more nucleotide sequences encoding tetherin can be replaced by a gene/nucleotide sequence of interest. The replaced gene/nucleotide sequence of interest can be the same or different, i.e. in one case, the same gene/nucleotide sequence of interest can be placed in the place of the nucleotide sequence encoding tetherin and in one another case, different genes/nucleotide sequences of interest can be placed in the place of the nucleotide sequence encoding tetherin. In one specific embodiment, the enveloped virus comprises two or more nucleotide sequences encoding Rousettus aegyptiacus or Desmodus retundus tetherin and the nucleotide sequences encoding Rousettus aegyptiacus or Desmodus retundus tetherin are replaced by a gene/nucleotide sequence of interest of the same type. In one another specific embodiment, the enveloped virus comprises two or more nucleotide sequences encoding Rousettus aegyptiacus or Desmodus retundus tetherin and the nucleotide sequences encoding Rousettus aegyptiacus or Desmodus retundus tetherin are replaced by a gene/nucleotide sequence of interest of different types.

The nucleotide sequence of interest may be selected from the group consisting of a sequence coding for an antigen, particularly an epitope of an antigen, a diagnostic compound, and a therapeutic compound. Preferably, the therapeutic compound is a virotherapeutic compound, a vaccine, or an oncolytic compound.

Specifically,
the cell line expressing a factor antagonizing tetherin is selected from the group consisting of an avian cell line, preferably a chicken retina cell line AGE1.CR.PIX, a DF-1 chicken fibroblast cell line, or a duck embryonic stem cell line EB66, and a mammalian cell line, preferably a baby hamster kidney (BHK) cell line or a human embryonal kidney 293 (HEK293) cell line, and/or
the cell line not expressing a factor antagonizing tetherin is selected from the group consisting of an avian cell line, preferably a chicken retina cell line AGE1.CR.PIX, a DF-1 chicken fibroblast cell line, or a duck embryonic stem cell line EB66, and a mammalian cell line, preferably a baby hamster kidney (BHK) cell line or a human embryonal kidney 293 (HEK293) cell line.

In one even more preferred embodiment,
the cell line expressing a factor antagonizing tetherin which is transfected in step (i) is an avian cell line, particularly a chicken retina cell line AGE1.CR.PIX, a DF-1 chicken fibroblast cell line, or a duck embryonic stem cell line EB66,
the factor antagonizing tetherin is a Tet Repressor (TetR) which modulates/is capable of modulating the activity of a Tet-Repressor (TetR) inducible promoter (e.g. a mH52dx promoter or a HYBdx promoter) controlling the expression of tetherin, and
the enveloped virus containing a first polynucleotide comprising a/at least one nucleotide sequence encoding tetherin is a vaccinia virus, such as a MVA virus, an MVA related virus, a vaccinia virus Dairen-I, or a vaccinia virus NYVAC, or an avipoxvirus, such as a canarypox virus (ALVAC) or a fowlpox virus.

Alternatively,
the cell line expressing a factor antagonizing tetherin which is transfected in step (i) is a mammalian cell line, particularly a baby hamster kidney (BHK) cell line or a human embryonal kidney 293 (HEK293) cell line,
the factor antagonizing tetherin is a Tet Repressor (TetR) which modulates/is capable of modulating the activity of a Tet-Repressor (TetR) inducible promoter (e.g. a mH52dx promoter or a HYBdx promoter) controlling the expression of tetherin, and
the enveloped virus containing a first polynucleotide comprising a/at least one nucleotide sequence encoding tetherin is a parapoxvirus, such as an ORF virus.

In one still even more preferred embodiment,
the cell line expressing a factor antagonizing tetherin which is transfected in step (i) is an avian cell line, particularly a chicken retina cell line AGE1.CR.PIX, a DF-1 chicken fibroblast cell line, or a duck embryonic stem cell line EB66,
the factor antagonizing tetherin is a Tet Repressor (TetR), and
the enveloped virus containing a first polynucleotide comprising a/at least one nucleotide sequence encoding tetherin is a Modified Vaccinia Ankara (MVA) virus or an MVA related virus,
wherein the/the at least one nucleotide sequence encoding tetherin is comprised in deletion site I, deletion site II, deletion site III, deletion site IV, deletion site V, deletion site VI, or tyrosine kinase deletion site of said virus, and/or
wherein the/the at least one nucleotide sequence encoding tetherin is under control of a Tet-Repressor (TetR) inducible promoter (e.g. a mH52dx promoter or a HYBdx promoter).

In one most preferred embodiment,
the cell line expressing a factor antagonizing tetherin which is transfected in step (i) is an avian cell line, particularly a chicken retina cell line AGE1.CR.PIX,
the factor antagonizing tetherin is a Tet Repressor (TetR),
the enveloped virus containing a first polynucleotide comprising a/at least one nucleotide sequence encoding tetherin is an MVA related virus,
wherein the/the at least one nucleotide sequence encoding tetherin is comprised in deletion site I, deletion site II, deletion site III, deletion site IV, deletion site V, deletion site VI, or tyrosine kinase deletion site of said virus, and
wherein the/the at least one nucleotide sequence encoding tetherin is under control of a Tet-Repressor (TetR) inducible promoter (e.g. a mH52dx promoter or a HYBdx promoter), and the tetherin is tetherin of chiropteran, particularly Rousettus aegyptiacus or Desmodus retundus.

In the embodiments, described above, the cell line infected in step (iii) is a cell line comprising a Tet Repressor (TetR) as factor antagonizing tetherin in which, however, TetR is deactivated by the addition of doxycycline (before/in step (iii)).

In a fifth aspect, the present invention relates to a pharmaceutical composition comprising an enveloped virus according to the first aspect.

The pharmaceutical composition comprising an enveloped virus according to the first aspect may comprise one or more excipient(s), diluent(s), and/or carrier(s), all of which are preferably pharmaceutically acceptable.

The pharmaceutical composition can be administered systemically, e.g. parenterally. For example, the pharmaceutical composition can be in a form suitable for oral administration, nasal administration, or administration by inhalation. The pharmaceutical composition can also be administered intravascular, intravenous, intramuscular, intrathecal, subcutaneous, or intraperitoneal.

The pharmaceutical composition can be administered in a single dose or in more than one dose. It is preferred that the pharmaceutical composition is to be administered in a therapeutically effective amount.

In a sixth aspect, the present invention relates to an enveloped virus according to the first aspect or a pharmaceutical composition according to the sixth aspect for use in medicine.

For example, the enveloped virus according to the first aspect or the pharmaceutical composition according to the sixth aspect is used in therapy such as oncotherapy, vaccination therapy, or gene therapy.

In a seventh aspect, the present invention relates to an enveloped virus according to the first aspect or a pharmaceutical composition according to the sixth aspect for use in therapy. Preferably, the therapy is oncotherapy, vaccination therapy, or gene therapy. More preferably, the therapy is oncotherapy.

For example, the virus may be used to suppress a transgene of therapeutic significance in a virus intended as a prophylactic or therapeutic vaccine, as a vehicle for gene therapy or molecular replacement, or as virotherapeutic or oncolytic agent.

The seventh aspect of the present invention can alternatively be worded as follows: A method for treating a subject comprising the step of: administering an enveloped virus according to the first aspect or a pharmaceutical composition according to the sixth aspect to a subject (in need thereof), thereby treating the subject.

The seventh aspect of the present invention can further alternatively be worded as follows: Use of an enveloped virus according to the first aspect or a pharmaceutical composition according to the sixth aspect for the manufacture of a medicament for treating a subject.

As mentioned above, attenuated viruses are viruses with compromised virulence in the intended recipient, e.g. human or animal recipient. Such a property can be achieved by adaptation of a virus to narrow temperature ranges or narrow host ranges and to other artificial replication environments, including chemically defined media. Attenuated viruses should not burden the host but stimulate its immune response. There are diverse attenuated viruses for prophylactic or therapeutic purposes available but many do not have the desired effect on the immune system.

Thus, there is an unmet need for new attenuated viruses having an improved effect on the immune system.

The present inventors have generated a new attenuated nucleocytoplasmic large DNA virus (NCLDV), wherein at least one essential gene has been replaced with the analogous essential gene of another NCLDV from a different virus family. Especially, the present inventors generated a vaccinia virus, wherein the genes coding for D9 and D10 of the vaccinia virus have been replaced with the (analogous) gene coding for g5R of an African swine fever virus (ASFV). D9 and D10 are decapping enzymes in the vaccinia virus that reduce accumulation of dsRNA, a potent trigger of innate immunity, in cells of a recipient. These decapping enzymes may even preferentially attack mRNAs important for innate immunity. The new mosaic virus that contains the gene coding for g5R in the vaccinia virus context causes a delayed but greater innate immune response which is beneficial to direct the immune system against intended targets such as tumors.

Accordingly, in an eight aspect, the present invention relates to a nucleocytoplasmic large DNA virus (NCLDV), wherein at least one essential gene (of said virus) has been replaced with the analogous essential gene of another NCLDV from a different virus family.

This aspect can alternatively be formulated as follows: A nucleocytoplasmic large DNA virus (NCLDV) comprising at least one essential gene of another NCLDV from a different virus family. This at least one essential gene replaces (the) originally existing analogous gene(s).

Specifically, the NCLDV/the other NCLDV is a virus of the Poxviridae or Asfarviridae family.

In one preferred embodiment,
the nucleocytoplasmic large DNA virus (NCLDV) is a virus of the Poxviridae family, wherein at least one essential gene (of said virus) has been replaced with the analogous essential gene of a virus of the Asfarviridae family, or
the nucleocytoplasmic large DNA virus (NCLDV) is a virus of the Asfarviridae family, wherein at least one essential gene (of said virus) has been replaced with the analogous essential gene of a virus of the Poxviridae family.

More specifically,
(i) the virus of the Poxviridae family is selected from the group consisting of a vaccinia virus, an avipoxvirus, and a parapoxvirus, or
(ii) virus of the Asfarviridae family is an African swine fever virus (ASFV).

Thus,
the NCLDV may be a vaccinia virus and the other NCLDV may be a virus of the Asfarviridae family such as an African swine fever virus (ASFV),
the NCLDV may be an avipoxvirus and the other NCLDV may be a virus of the Asfarviridae family such as an African swine fever virus (ASFV), or
the NCLDV may be a parapoxvirus and the other NCLDV may be a virus of the Asfarviridae family such as an African swine fever virus (ASFV).

Alternatively, the
the NCLDV may be a virus of the Asfarviridae family such as an African swine fever virus (ASFV) and the other NCLDV may be a vaccinia virus,
the NCLDV may be a virus of the Asfarviridae family such as an African swine fever virus (ASFV) and the other NCLDV may be an avipoxvirus, or
the NCLDV may be a virus of the Asfarviridae family such as an African swine fever virus (ASFV) and the other NCLDV may be a parapoxvirus.

In one more preferred embodiment,
the nucleocytoplasmic large DNA virus (NCLDV) is a vaccinia virus, wherein at least one essential gene (of said virus) has been replaced with the analogous essential gene of an African swine fever virus (ASFV), or
the nucleocytoplasmic large DNA virus (NCLDV) is an African swine fever virus (ASFV), wherein at least one essential gene (of said virus) has been replaced with the analogous essential gene of a vaccinia virus.

Even more specifically,
(i) the vaccinia virus is an attenuated vaccinia virus selected from the group consisting of a Modified Vaccinia Ankara (MVA) virus, an MVA related virus, a vaccinia virus Dairen-I, and a vaccinia virus strain NYVAC,
(ii) the avipoxvirus is a virus selected from the group consisting of a canarypox virus and a fowlpox virus, or
(iii) the parapoxvirus is an ORF virus.

Thus,
the NCLDV may be a Modified Vaccinia Ankara (MVA) virus, and the other NCLDV may be a virus of the Asfarviridae family such as an African swine fever virus (ASFV),
the NCLDV may be an MVA related virus and the other NCLDV may be a virus of the Asfarviridae family such as an African swine fever virus (ASFV),
the NCLDV may be a vaccinia virus Dairen-I and the other NCLDV may be a virus of the Asfarviridae family such as an African swine fever virus (ASFV), or
the NCLDV may be a vaccinia virus strain NYVAC and the other NCLDV may be a virus of the Asfarviridae family such as an African swine fever virus (ASFV).

Alternatively, the
the NCLDV may be a virus of the Asfarviridae family such as an African swine fever virus (ASFV) and the other NCLDV may be a Modified Vaccinia Ankara (MVA) virus,
the NCLDV may be a virus of the Asfarviridae family such as an African swine fever virus (ASFV) and the other NCLDV may be an MVA related virus,
the NCLDV may be a virus of the Asfarviridae family such as an African swine fever virus (ASFV) and the other NCLDV may be a vaccinia virus Dairen-I, or
the NCLDV may be a virus of the Asfarviridae family such as an African swine fever virus (ASFV) and the other NCLDV may be a vaccinia virus strain NYVAC.

Thus,
the NCLDV may be a canarypox virus, and the other NCLDV may be a virus of the Asfarviridae family such as an African swine fever virus (ASFV), or
the NCLDV may be a fowlpox virus, and the other NCLDV may be a virus of the Asfarviridae family such as an African swine fever virus (ASFV).

Alternatively, the
the NCLDV may be a virus of the Asfarviridae family such as an African swine fever virus (ASFV) and the other NCLDV may be a canarypox virus, or
the NCLDV may be a virus of the Asfarviridae family such as an African swine fever virus (ASFV) and the other NCLDV may be a fowlpox virus.

Thus, the NCLDV may be an ORF virus, and the other NCLDV may be a virus of the Asfarviridae family such as an African swine fever virus (ASFV).

Alternatively, the NCLDV may be a virus of the Asfarviridae family such as an African swine fever virus (ASFV) and the other NCLDV may be an ORF virus.

In one even more preferred embodiment,
the nucleocytoplasmic large DNA virus (NCLDV) is a Modified Vaccinia Ankara (MVA) virus or an MVA related virus, wherein at least one essential gene (of said virus) has been replaced with the analogous essential gene of an African swine fever virus (ASFV), or
the nucleocytoplasmic large DNA virus (NCLDV) is an African swine fever virus (ASFV), wherein at least one essential gene (of said virus) has been replaced with the analogous essential gene of a Modified Vaccinia Ankara (MVA) virus or an MVA related virus.

The at least one essential gene may be selected from the group consisting of a gene coding for
(i) an enzyme required for genome replication,
(ii) a defensive gene product, and/or
(iii) an enzyme required for morphogenesis.

Preferably,
the enzyme required for genome replication is a Holliday junction resolvase (A22), uracil-DNA glycosylase (D4), or poly-A polymerase subunit (D7, J3), and/or
the defensive gene product is a decapping enzyme, inhibitor of cGAS signaling (E5), or inhibitor of interferon signaling (E3), and/or
the enzyme required for morphogenesis is a core protein protease (I3), a core protein (P4a, P4b, P25K), or scaffold assembly factor (D13).

More preferably, the decapping enzyme is selected from the group consisting of D9 of a vaccinia virus, D10 of a vaccinia virus, and g5R of an African swine fever virus (ASFV).

In this respect, it should be noted that the D9 protein is encoded by the D9R gene (also designated as MVA106R), the D10 protein is encoded by the D10R gene (also designated as MVA107R), or the g5R protein is encoded by the D250R gene.

Still even more preferably,
the nucleocytoplasmic large DNA virus (NCLDV) is a vaccinia virus, wherein the genes coding for D9 and D10 (of said virus) have been replaced with the gene coding for g5R of an African swine fever virus (ASFV), or
the nucleocytoplasmic large DNA virus (NCLDV) is an African swine fever virus (ASFV), wherein the gene coding for g5R (of said virus) has been replaced by the genes coding for D9 and D10 of a vaccinia virus.

Most preferably,
the nucleocytoplasmic large DNA virus (NCLDV) is an MVA virus or an MVA related virus, wherein the genes coding for D9 and D10 (of said virus) have been replaced with the gene coding for g5R of an African swine fever virus (ASFV), or
the nucleocytoplasmic large DNA virus (NCLDV) is an African swine fever virus (ASFV), wherein the gene coding for g5R (of said virus) has been replaced by the genes coding for D9 and D10 of an MVA virus or an MVA related virus.

The Genbank Accession number of the MVA related virus is KY633487 (version KY633487.1). The position of the coding sequence for D9R (or MVA106R) is 110634-111275 and the position of the coding sequence for D10R (or MVA107R) is 111272-112018. Thus, in case of the MVA related virus, it is specifically preferred that the nucleotide sequence encompassing nucleotides 110634 to 112018 has been replaced by D250R.

All replacements as described above have been carried out via homologous recombination (see experimental section).

Especially, the NCLDV further comprises a (heterologous) nucleotide sequences of interest.

The position suitable for the incorporation of a (heterologous) nucleotide sequence of interest is selected from the group consisting of natural deletion sites and intergenic sites.

In case of a MVA virus, this position is preferably selected from the group consisting of deletion site I, deletion site II, deletion site III, deletion site IV, deletion site V, deletion site VI, between two essential or non-essential genes or replacement of one or more non-essential gene(s) including the tyrosine kinase gene.

In case of an MVA related virus, this position is preferably selected from the group consisting of deletion site II, deletion site III, deletion size IV, deletion site V, deletion size IV, between two essential or non-essential genes or replacement of one or more non-essential gene(s) including the tyrosine kinase gene.

More preferably, the (heterologous) nucleotide sequence of interest is selected from the group consisting of a sequence coding for an antigen, particularly an epitope of an antigen, a diagnostic compound, and a therapeutic compound. More preferably, the therapeutic compound is a virotherapeutic compound, a vaccine, an immunomodulatory compound, preferably interleukin 2, or an oncolytic compound.

In a ninth aspect, the present invention relates to a pharmaceutical composition comprising a nucleocytoplasmic large DNA virus (NCLDV) according to the eight aspect.

The composition may comprise one or more pharmaceutically acceptable excipients, diluents, and/or carriers.

The pharmaceutical composition can be administered systemically, e.g. parenterally. For example, the pharmaceutical composition can be in a form suitable for oral administration, nasal administration, or administration by inhalation. The pharmaceutical composition can also be administered intravascular, intravenous, intramuscular, intrathecal, subcutaneous, or intraperitoneal.

The pharmaceutical composition can be administered in a single dose or in more than one dose. It is preferred that the pharmaceutical composition is to be administered in a therapeutically effective amount.

In a tenth aspect, the present invention relates to a nucleocytoplasmic large DNA virus (NCLDV) according to the eight aspect or a pharmaceutical composition according to the ninth aspect for use in medicine.

For example, the NCLDV according to the eight aspect or the pharmaceutical composition according to the ninth aspect is used in therapy such as oncotherapy, vaccination therapy, or gene therapy.

In an eleventh aspect, the present invention relates to a nucleocytoplasmic large DNA virus (NCLDV) according to the eight aspect or a pharmaceutical composition according to the ninth aspect for use in therapy.

Preferably, the therapy is oncotherapy, vaccination therapy, or gene therapy.

The eleventh aspect of the present invention can alternatively be worded as follows: A method for treating a subject comprising the step of: administering a NCLDV according to the eight aspect or a pharmaceutical composition according to the ninth aspect to a subject (in need thereof), thereby treating the subject.

The eleventh aspect of the present invention can further alternatively be worded as follows: Use of a NCLDV according to the eight aspect or a pharmaceutical composition according to the ninth aspect for the manufacture of a medicament for treating a subject.

The above-mentioned subject may be a mammal such as a human. The subject may also be an animal.

For applications where coinfection with a helper virus is required (for example, to rescue a recombinant virus out genomic DNA provided as artificial chromosome), the present invention further describes an attenuation (weakening) method by replacing viral defensive genes of one nucleocytoplasmic large DNA virus (NCLDV) with those from a related NCLDV. This replacement leads to a mosaic virus with a mismatch in the genotype that leads to impaired replication. The approach maintains the original cell line dependence so that no change of cellular substrate is required. A recombination with a gene of interest flanked by the original (cognate) viral defensive genes removes the newly introduced immune factor and promotes replication of desired recombinants.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIG. 1: Genomic organization of wild-type MVA and strain CR19. The terminal repeats have expanded in size in MVA-CR19 by a recombination event that has caused loss of deletion site (DS) I at the left side and duplication of DS IV at both sides of the genome. In this example, tetherin has been inserted in DS IV. It can be inserted into any other DS or intergenic site.

FIG. 2: TetR supports and doxycycline inhibits replication of well-designed tMVAs. Wild-type MVA and strain CR19 that carry tetherin under control of an inducible HYB or mH5 promoter in DS IV replicate in presence of TetR designed to bind to and block the promoter. In the presence of doxycycline the promoter is de-blocked, tetherin can be expressed and tMVA does not replicate (decline of red signals).

FIG. 3: TetR supports (open symbols) and de-blocking by addition of doxycycline inhibits replication (filled symbols) of tMVAs.

FIG. 4: Addition of doxycycline has clear effects on replication of tetherin-inducible viruses even if added 24 h post infection. Both, number (left panel) and size of plaques (right panel) are reduced compared to the uninhibited control (-).

FIG. 5: appearance of infected cultures that are quantified in FIG. 4 under doxycycline de-blocking.

FIG. 6: The tetherin expression cassette is replaced within one passage. Successful replacement of the tetherin-containing insertion in DS IV by the dual-expression cassette in the shuttle plasmid for same DS IV results in a loss of tetherin expression and gain of EGFP expression. The constitutive expression of mCherry is maintained. In the presence of doxycycline tetherin expression is possible and the parental virus is depleted. Without doxycycline the parental (tetherin-containing) virus can replicate. The upper panel in the Passage 1 column is an impressive demonstration of the low probability of recombination (green plaque) in the background of parental virus (red without green).

FIG. 7: Tetherin expression cassette is replaced within one passage. The expected result by automated quantification is an increase of EGFP expression towards a relative ratio of 0.5. A slight bias towards EGFP expression can be observed because maturation of this fluorescent marker is faster than maturation of mCherry and because the viral promoter for EGFP is shifted towards earlier time points relative to the late P11 promoter that drives mCherry. The difference between enrichment of the desired recombinant is clear: all passage 2 populations (independent of whether they are selected at passage 2 or not, both groups had been selected at passage 1) exhibit the EGFP:mCherry 1:1 phenotype of successful recombination. The quantification also demonstrates successful rapid gene swapping for the two promoters, HYBdx and mH52dx.

FIG. 8: Genotype development during recovery of MVA-G5R. PCR shown here was performed on isolates out of purification round 2, 5, 10 and 12. The three arrows point to the position of expected amplicons for MVA-G5R viruses that contain the EGFP transient marker (2296 bp), MVA-CR19 parental virus (1742 bp) and the desired MVA-G5R vector without transient marker (1219 bp). A complete depletion of parental MVA-CR19 was achieved with isolate 13 in purification round 12.

FIG. 9: MVA-G5R is pure (without wildtype contamination) and shown here with two inserts at different insertion sites (DS III and DS IV). PCR was done with genomic DNA isolated from cells infected with MVA-G5R. Note that no DS I signal is visible because MVA-G5R is derived from MVA-CR19. In wildtype MVA the expected signal would be 291 bp. The other confirmed amplicons are 1099 bp for 4PT-RED in DS IV (220 bp for empty DS IV), 702 bp for wildtype DS VI, 1219 bp for D9/D10 replaced by g5R (1742 bp in parental MVA), and 1285 bp for 3EL-GFP in DS III (446 bp for wildtype DS III).

FIG. 10: MVA-G5R replicates in anatine adherent and suspension cultures. Shown are adherent cultures 72 h and suspension cultures 96 h post infection. Cytopathic effect of MVA-G5R in adherent CR.pIX is weak also 72 h post infection although expression of reporters is strong. Similarly, although spread of MVA-G5R through the aggregates is robust and expression of reporters is strong the cytopathic effect is delayed beyond 96 h post infection also in the suspension cells.

FIG. 11: tCR9 helper cells show gene regulation for different constructs of tetherin-expressing viruses and a control virus that expresses EGFP from a switchable promoter in a cassette that runs antiparallel to the gene for mCherry. The experiment was visualised with a NyOne Scientific (Synentec) plate scanner. The image tiles for individual channels were inverted and contrast-enhanced with Affinity Photo (Serif Europe Ltd) for improved presentation as non-colored figures.

FIG. 12: A) Normal CR pIX cells (cells that do not express TetR) were stably transfected for expression of an EGFP-Dr-tetherin fusion protein. These cells limit replication of normal MVA, although not to levels seen with tetherin-expressing MVAs. The upper panel shows the EGFP-Dr-tetherin positive cells (visible by the general EGFP background). The lower panel shows normal (parental) CR pIX cells. Viruses that express tetherin (either Ra, Dr or a GFP-Dr-tetherin fusion) are strongly inhibited. The virus that expresses the GFP-Dr-tetherin fusion appears to be inhibited to a lower degree compared to the other viruses. B) Subcellular distribution of the EGFP-Dr-tetherin protein in the stably transfected CR pIX cell lines. See legend in FIG. 11 about image enhancement.

FIG. 13: Fluorescence patterns of MVA-CR19 that encodes Ra tetherin and mTagBFP2 in DS VI and Dr tetherin and mCherry in the TK locus. See legend in FIG. 11 about image enhancement. (A). Genetic confirmation of tetherins in DS VI and the TK locus by PCR against the insertion sites.

FIG. 14: Simultaneous (concurrent) replacement of the tetherins in the two distant insertion sites by two EGFP and mCherry dual expression cassettes. The dual tetherin virus (ttMVA) is the one shown in FIG. 13. See legend in FIG. 11 about image enhancement.

FIG. 15: Genetic confirmation of the double concurrent replacement by PCR. Three separate wells of a 6-well plates were infected with passage-1 lysate. All wells gave similar results, shown here is the result obtained with well 2 (w2 in the figure). Results for amplification of the complete insertion site (TK locus or DS VI) are shown in the left section of each gel. A confirmation for replacement of the tetherin marker (absence of tetherin) in the desired recombinants is shown in the right section of each gel. The desired signals are 2591 bp for the new inserts in the TK locus and 2195 bp in DS VI. The parental tetherin-containing insertion yields an amplicon of 2525 bp for the TK locus and 2035 bp for DS VI. The expected amplicon for the native TK locus is 754 bp and for DS VI is 406 bp. Unspecific background amplification is shown in the lanes marked with tCR9 where DNA from non-infected cells was used as template.

FIG. 16: Rapid generation of recombinant viruses using a suspension cell process in a chemically defined culture environment without animal derived components. Panel (A) illustrates infection followed by transfection in suspension and adherent cultures (passage 0, P0). Tetherin expression was suppressed (“no dox”) or allowed 3 h and 17 h post infection as indicated. Without fluorescent or marker-assisted cell sorting (suspension cells) or plaque-picking (adherent culture), fresh suspension (B) or adherent (C) tCR9 cells, respectively, were infected with complete lysates of the P0 cultures. The infected cultures were treated with dox to allow expression of tetherin or left without dox. The desired outcome is expression of both EGFP and mCherry, as seen in columns 1 each of panels (B) and (C). Presence of red-only foci indicates undesired presence of parental virus and can be seen in tCR9 cells that repress tetherin expression—for example in columns 2 each of panels (B) and (C). The images have been inverted and each channel is shown separately for improved visibility in non-color representations.

### EXAMPLES

The examples given below are for illustrative purposes only and do not limit the invention described above in any way.

### Example 1

### Design of Shuttle Vectors

The shuttle vector pSh III ELP11 Dual for deletion site (DS) III has been described previously (Jordan et al. 2019). The shuttle vectors described here are derivatives therefrom. The flanks were obtained by PCR on viral genomic DNA or by direct cloning of synthetic DNA. Insertion into the backbone of the DS III shuttle vector was done with restriction sites PciI, NheI, NotI or DraIII that were contained in the outer primers or the synthetic DNA. PCR primers (where applicable) and expected sizes of the flanks are shown in the table. Sequence of all PCR-amplified regions was confirmed by Sanger sequencing of the final plasmids.

The primers used for cloning of the left flank 820 bp) via PciI and NheI for the shuttle plasmid for DS IV are SEQ ID NO: 1 and SEQ ID NO: 2, and for cloning of the right flank (888 bp) via NotI and DraIII are SEQ ID NO: 3 and SEQ ID NO: 4.

The primers used for cloning of the left flank (847 bp) via PciI and NheI for the shuttle plasmid for DS VI are SEQ ID NO: 5 and SEQ ID NO: 6, and for cloning of the right flank (919 bp) via NotI and DraIII are SEQ ID NO: 7 and SEQ ID NO: 8.

The primers used for cloning of the left flank (670 bp) via DraIII and NotI for the shuttle plasmid for DS V are SEQ ID NO: 9 and SEQ ID NO: 10, and for cloning of the right flank (659 bp) via NheI and PciI are SEQ ID NO: 11 and SEQ ID NO: 12.

The recombination flanks for the shuttle plasmid for the TK locus were fully synthesized (GeneArt) with the appropriate restriction enzyme sites for the left flank (PciI and NheI) and right flank (Not and DraIII). The flanks had a size of 721 bp (from gttttatttg to attctttatt) and 685 bp (atgaacggcg to agtgcaaatg) on the viral genomic DNA.

Screening for the insertion in DS IV was done with primers SEQ ID NO: 13 and SEQ ID NO: 14. The sizes are 220 bp in wildtype MVA, 2016 bp for the dual expression construct, 1231 bp for the construct with GFP and 1099 bp with mCherry.

Screening for the insertion in DS VI was done with primers SEQ ID NO: 15 and SEQ ID NO: 16.

Each shuttle vector allows replacement of the complete payload within the recombination flanks with NheI and NotI. The promoter at the left side can be replaced with NheI and BglII or XhoI.

Cellular and viral thymidine kinases (TKs) augment availability of the dTTP pool required for DNA synthesis. The poxviral TK is not essential for replication but poxviruses were shown to be attenuated if expression of this enzyme is impaired. The TK shuttle plasmid for generation of MVA intended for vaccine development was therefore designed to maintain thymidine kinase expression. Consensus elements of early promoters (Yang et al. 2011) can be found in a sequence that is 19 bp upstream of the TK gene (MVA086R, J2R in VACV Copenhagen, position 87347-87880 of Genbank KY633487 for MVA-CR19). This potential promoter is duplicated also at the start of the proximal flank to allow a hybrid early/mH5 expression for transgenes. The sequence of the TK UTR was shuffled at the distal flank to reduce homologies caused by the repeat of the presumed TK promoter within the flank that may promote deletion of the insert.

### Example 2

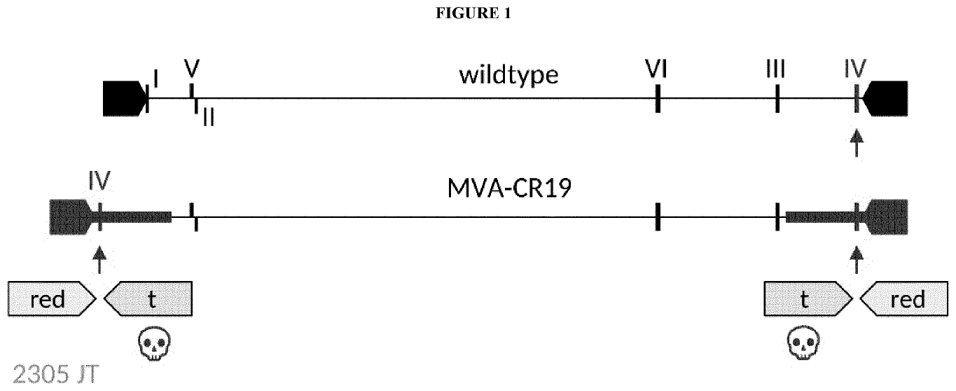

Generation of a Tetherin MVA (tMVA) Expression Cassette Generation of recombinant viruses depends on an adherent cell culture phase in media supplemented with animal-derived serum. Isolation of plaques furthermore requires reliable means of identification of desired viruses by their phenotype in host cells and physical access to the infected monolayer. As demonstrated in the previous study, viruses of different lineages may be inadvertently obtained from neighbouring plaques or even from a single plaque after undesired co-infection with virion aggregates. A method not plagued by these limitations would be highly advantageous especially for pandemic preparedness and personalised treatments that depend on compressed timelines between identification of a target and provision of recombinant vectors. A novel toxic marker (the innate immune factor tetherin) was tested for these purposes in a challenging position of the viral genome, the terminal repeat, as a proof of concept (FIG. 1). The system was subsequently shown to be functional in other sites of the genomic, including the core region.

Tetherin was inserted as synthetic DNA designed in two steps. First, Rousettus aegyptiacus (Ra) tetherin (Genbank XM_016157800) was reverse translated from the protein sequence into a DNA sequence with codon preference for Homo sapiens. In a second step, repeats of 5 or more T-nucleotides were removed as these may function as terminator signals for vaccinia virus early transcription cassettes (Yuen and Moss 1987). This optimization changed 135 bp in the ORF of 552 bp (184 amino acids). The nucleotide sequence of this optimised Ra tetherin is given in SEQ ID NO: 17.

Opposing vaccinia virus terminator sequences were added downstream of the coding sequence and sites for the restriction enzymes BamHI, NotI and SwaI were included for subsequent cloning steps. This optimized tetherin construct was synthesized by Life Technologies (GeneArt) and inserted into a shuttle vector for deletion site IV of MVA and MVA-CR19. This shuttle vector (p45T-Teth-RED) expresses Ra tetherin from the mH5 promoter and mCherry reporter protein from the P11 late promoter.

The tetherin shuttle vector for insertion into the thymdine kinase (TK) site was obtained by insertion of the tetherin/mCherry expression cassette as NheI/NotI fragment from an earlier shuttle vector into the same sites of synthetic DNA (p22ABZAYP_3338946_qTK-Zero). The synthetic DNA was designed with 721 bp of a left flank that contained the gene MVA085R (position 74833 to 75553 in MVA Genbank U94848 or 86620 to 87340 of MVA-CR19 Genbank KY633487) and 685 bp of a right flank that contained the gene MVA086R (position 75559 to 76243 in MVA Genbank U94848 or 87346 to 88030 of MVA-CR19 Genbank KY633487). Gene MVA086R is the thymidine kinase (similar to vaccinia Copenhagen J2R). The sequence between left and right flanks was designed to maintain the potential thymidine kinase promoter; followed by NheI, BsrGI and NotI as restriction enzyme sites for insertion of expression cassettes; followed by an early MVA terminator; followed by a stretch of 23 bp that contains the potential TK promoter and therefore is homologous to the end of the left flank; followed by a 12 bp UTR that has been changed in such a way that further homology to the left flank is avoided but GC content is maintained with exception of the terminal C in an attempt to improve the Kozak environment upstream of TK.

### Example 3

Generation of a Suppressor Cell Line (tCR9)

MVA-CR19 appears to be released more easily from the infected host cell than wild-type MVA (Jordan et al. 2013). It was hoped to reduce this mobility of CR19 to wild-type levels or even below by expression of tetherin out of selected genomic loci. Replacement of tetherin by a gene of insert via recombination would reconstitute the CR19 phenotype and thus improve rescue of recombinants from the supernatant (rather than complete lysate) of infected cultures. However, Ra tetherin could not be inserted into MVA-CR19 or wild-type at all. The initial experiment was repeated twice with S33 p45T-TethRED, a shuttle plasmid for DS IV that drives expression of tetherin with the mH5 promoter. This observation is surprising because tetherins are reported not to be active against poxviruses (Sliva et al. 2012). Furthermore, tetherin from the Rousette bat was inserted. Cell lines from this bat were shown to be permissive for MVA (Jordan et al. 2009) so that any activity of that tetherin is even more surprising.

Thus, the activity of the toxin must be efficiently suppressed in a suitable host cell line to obtain viruses that carry the marker for subsequent further manipulation. A partial or complete block is also required during recombination because generation of novel progeny viruses requires at least transient genomic replication. However, depletion of the parental (tetherin-expressing) virus depends on the full activity of the toxic marker.

One desired controlled suppression of the toxic marker was achieved using the Tet-repressor (TetR) technology. As a first step, a eukaryotic expression plasmid was generated where TetR was inserted under control of the human CMV promoter. The coding sequence for TetR was optimized for the human codon frequency and (because vaccinia viruses replicate in the cytoplasm) did not contain a nuclear localisation sequence (NLS) as opposed to frequently used TetR systems. The expression of the mRNA for TetR was linked via an internal ribosome entry sequence (IRES) to the puromycin resistance gene. An SV40 polyadenylation signal was used to terminate the transcription. Downstream of the polyA signal a mouse U6 promoter for polymerase III was inserted, in antiparallel orientation, for expression of an shRNA directed against a target within the codon-optimized tetherin gene in MVA.

The TetR-expression plasmid was linearized with SspI and purified with the Wizard® SV Gel and PCR Clean-Up System (Promega). CR.pIX cells were seeded at 0.5E6 viable cells/ml in a 6 well plate and transfected on the following day with 2 μg of the purified plasmid with the Effectene® Transfection Reagent (200 μL buffer, 16 μL enhancer, 20 μL effectene). Medium was replaced 16 h post transfection. Puromycin for selection was added 48 h post transfection at 1 μg/mL and was maintained throughout. The resulting helper cell line that allows replication of tMVA is called tCR9. To obtain tCR9 suspension cell lines, a GMP-grade working cell bank-derived preparation of suspension CR.pIX cells were transiently cultivated in an adherent format in DMEM:F12 containing gamma-irradiated 5% FCS of certified and documented negligible risk TSE origin. The same transfection protocol was then applied as described for the adherent cell line. After several passages, this tCR9 culture was shifted to chemically-defined suspension CD-U7 medium (Xell) and further cultivated in shake flasks.

Tetherin-containing MVAs (tMVAs) were generated in tCR9 cells by homologous recombination. First, 0.5e6 to 1e6 adherent tCR9 cells were seeded per well of a 6-well plate and incubated overnight. On the following day, the cell monolayer was infected with MVA (wildtype or CR19) with a multiplicity of 0.01. After 90 minutes, the infected cells were liposomally transfected with shuttle plasmids for tetherin (Examples 1 and 2) using Effectene as described above. The transfection medium was replaced after 4-16 h, and 48 h post infection cells were dislodged by pipetting and lysed with ultrasound treatment in a VialTweeter (Hielscher) for 20 s with 100% cycle and 90% amplitude. Fresh tCR9 cell monolayers in a 6-well plate were infected with the 48 h-lysate of the infection/transfection at a dilution of 1:1000-1:50000. The culture medium was replaced with 1% methylcellulose (Sigma) in DMEM:F12 (Gibco) and 5% FCS after 2-4 h. Plaques were aspirated as a volume of approx. 20 μL with a 100 μL pipette tip, dissolved in 0.5 mL of culture medium and sonicated. 50-100 μL therefrom was transferred to a fresh monolayer of tCR9 cells in a 6-well plate without further dilution. Methylcellulose overlay was applied after 2-4 h and plaque purification repeated until parental virus could not be detected anymore by PCR.

For PCR, total DNA was recovered from a 20 μL aliquot of the lysate of a purification step by adding 5 μL of QuickExtract™ DNA Extraction Solution (Cambio, UK) and heating to 65° C. for 10 min and 98° C. for 5 min. 5 μL therefrom was used in a PCR reaction with final volume of 25 μL. PCR primers were chosen to amplify the complete insertion cassette including portions of the recombination flanks so that contamination by wildtype and due to partial deletions can be observed.

Fully plaque-purified tMVA viruses were amplified once on adherent tCR9 cells in 6-well plates prior to production of larger stocks in suspension tCR9 cultures as described previously (Jordan et al. 2013). Genomic DNA was isolated with the Qiagen Blood purification kit according to the manufacturer's instructions for detailed analysis.

### Example 4

### Promoter Optimization for Balanced Expression of the Toxic Marker

Several different viral promoters with tetO-tandem repeats were next designed for inducible suppression via TetR that is expressed in the tCR9 helper cell line. Different promoters were tested because the expression strength and the position of the TetO2 sites relative to the (putative) transcription start site are important parameters for the level of promoter attenuation. The tested promoters include a synthetic hybrid promoter that is similar to an earlier design (Chakrabarti et al. 1997) but differs in an core residue (A instead of G) (Yang et al. 2011; Jordan et al. 2019) and the mH5 promoter (Wyatt et al. 1996). The canonical tetO sequence was used as a tandem repeat separated by a tc or ct dinucleotide. The promoter sequences are SEQ ID NO: 18 and SEQ ID NO: 19.

mCherry driven by the late P11 promoter was inserted antiparallel to the upstream ORF (either tetherin or EGFP) in the same cassette as a control for viability of the viruses. The different promoters were tested by recombination of the tetherin/mCherry dual cassette into DS IV of wildtype and strain CR19 viruses. They were also inserted into DS V, DS VI and downstream of thymdine kinase (TK, gene MVA086R in Genbank U94848).

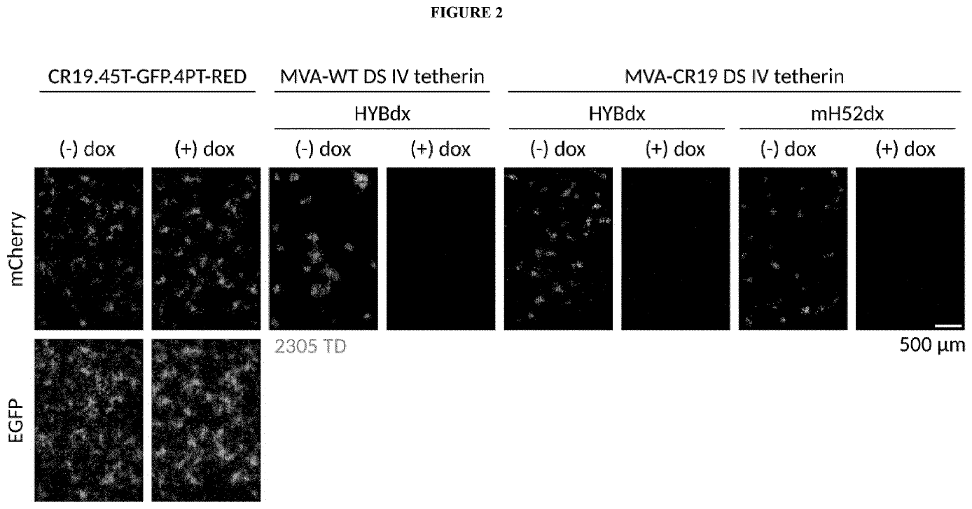

Suppression of expression in presence of TetR allowed replication of tMVAs that carried tetherin under control of the mH52dx and HYBdx promoters. Addition of doxycyclin restored tetherin expression and interfered with tMVA replication to such an extent that independent mCherry reporter expression was not detectable (FIG. 2). Replication of MVA-CR19 that expresses EGFP and mCherry out DS IV was not affected by addition of doxycycline.

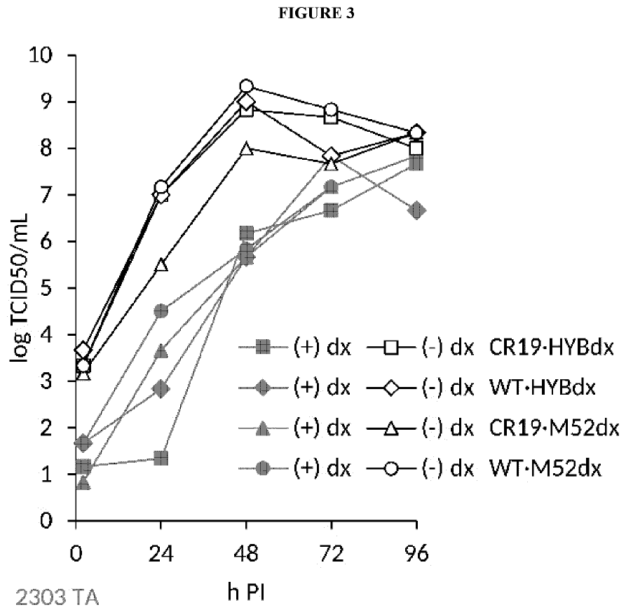

Effect of tetR repression is also visible in the replication kinetic in industrial relevant suspension cultures of wild-type and strain CR19 MVAs that carry tetherin under control of the HYBdx and mH52dx promoters (FIG. 3). Whereas the tCR9 suspension cell supported replication of viruses with tetherin in DS IV to expected levels of at least 10\hat{ }8 TCID50/mL, addition of doxycycline removed the block on tetherin expression and reduced titers of the same viruses 200 to 3000-fold.

This experiment demonstrated that generation of tMVA is possible if a combination of requirements is met. The requirements include careful selection of a toxic marker (here, Ra tetherin), careful adjustment of the expression strength of the viral promoter for the marker, design of a suitable system for conditional expression, and a helper cell line that that is fully permissive for the wildtype MVA but also can suppress the ectopic tetherin activity.

### Example 5

### Dox Response Kinetic

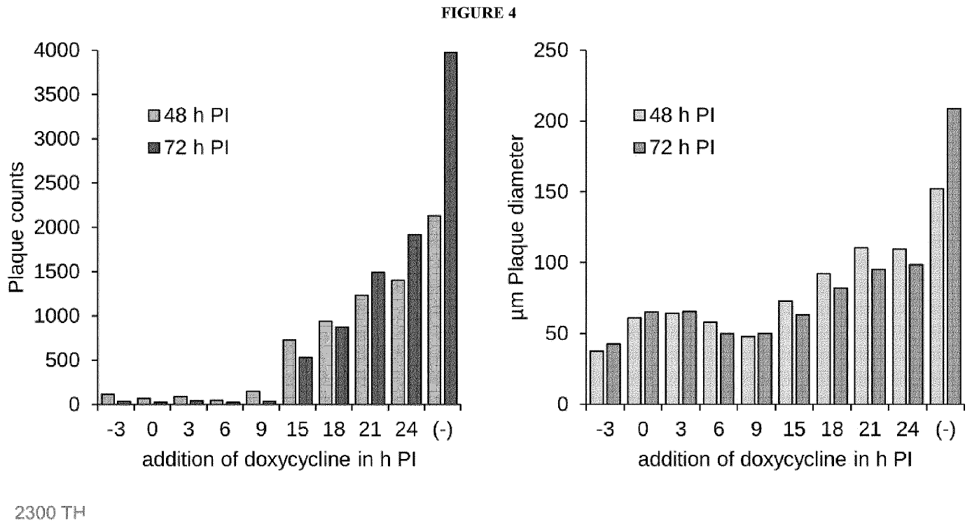

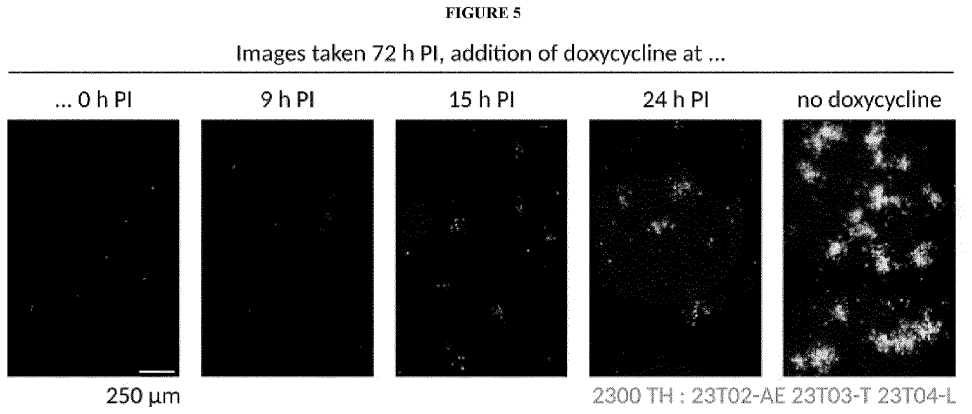

The protocol is furthermore robust and resilient against timing of the workflow. Replication of MVA-CR19.4HYBdxT-Teth-RED was prevented if doxycycline was added up to 9 h post infection (FIG. 4 and FIG. 5). Even if doxycycline was added 24 h post infection a weak red fluorescence signal was visible in cells. The weak signal was consistent with abortive infection and the frequency consistent with an input MOI of 0.01.

### Example 6

Functional Assay of Recombination with tMVA

A functional selection against parental viruses was first tested with tMVA vectors that contain the marker in DS IV under control of the doxycycline-inducible mH5 or HYB promoters (MVA-CR19.4M52dxT-Teth-RED and MVA-CR19.4HYBdxT-Teth-RED). The titers of the two receiving tMVA vectors were 3.25e7 (mH52dx design) and 4.77e8 (HYBdx) PFU/mL. Cultures of 1e6 tCR9 cells per well of a 6-well-plate were infected with MOI of 0.01 and transfected with 2 μg each of p45T-Dual shuttle plasmid 90 minutes after infection. Transfection was performed with Effectene Transfection Reagent (Qiagen) as described above. The infection/transfection (passage 0) and subsequent passages were done without de-repression, or with doxycyclin added to 1 μg/mL to study the impact of tetherin expression on the selection process. Complete lysates of passage 0 were obtained after 48 h.

Addition of doxycycline during recombination severely reduced the number of potential recombinants, consistent with the concept that recombination requires replication of the receiving virus.

For quantification of the efficacy of recombination, a complete lysate of passage 0 that did not receive doxycycline was used to infect tCR9 monolayers in 12-well plates (passage 1). Half of the wells were given doxycycline, the other half was kept with repression of tetherin expression. Of each format, some wells received medium with methylcellulose 2 h post transfection to facilitate quantification of plaques. Plaques were counted by NyOne (Synentec) scanning and analysis with the Fluorescent Plaque Morphology (2F) (v. 0.9) module 72 h post infection. Wells without methylcellulose that received doxycycline were harvested and used for infection of fresh tCR9 monolayers in 12-well-plates for passage 2. Passage 2 wells also received methylcellulose for quantification and no methylcellulose for generation of lysate.

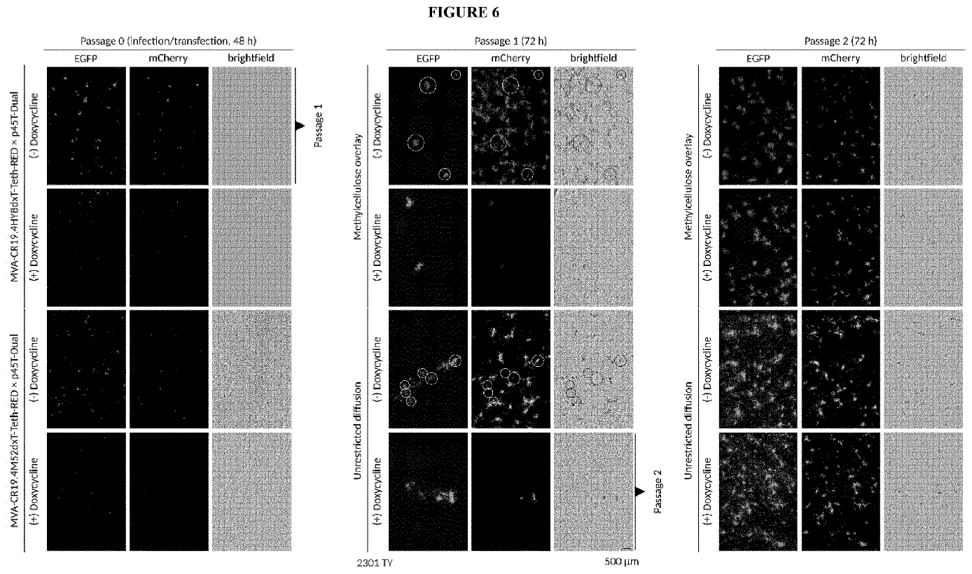

The results are shown in FIG. 6. In both cultures (with or without methylcellulose overlay) that did not receive doxycycline the number of red plaques exceeds the number of green plaques more than 20-fold (under methylcellulose) or 3-5 fold (if virion diffusion is not obstructed). The reason for this difference may be due to a restriction of MVA-CR19 comet formation by tetherin. The difference in the phenotype for tMVA-CR19 and MVA-CR19 that does not express tetherin may be more profound if no methylcellulose overlay is applied. The GFP signals in comets may be overrepresented in the quantification even if they are derived only from few founding plaques. This overrepresentation may bias against the actual number of red signals of tMVA.

No non-fluorescent revertants were visible in the brightfield images. This observation is consistent with the high efficiency of the recombination towards the desired genotype. It is also a sign that regulation of tetherin was achieved to an extent that is sufficient for prevention of emergence of revertants.

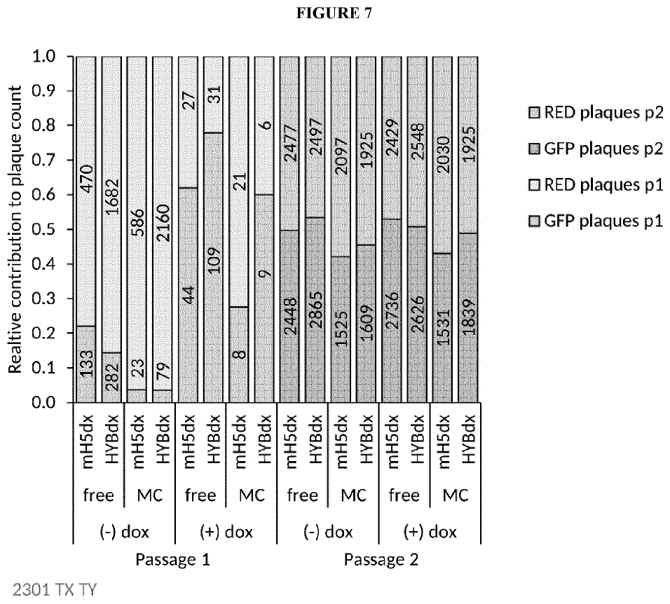

In presence of doxycycline a lower number of plaques was observed. However, the lower number of plaques may be a reflection of the sporadicity of actual recombination events. Replacing the tetherin and mCherry dual expression cassette with a EGFP and mCherry dual expression cassette can be observed only by gain of GFP expression, not by loss of mCherry expression. In a culture infected with recombined viruses equivalent numbers of green and red plaques therefore are expected. This value is approached in the presence of doxycycline starting with passage 1 and confirmed at passage 2 that here was derived out of the doxycycline-selected passage 1. No further effect by doxycycline was visible at passage 2 suggesting a maximal efficacy already within a single passage and without the need for plaque picking (FIG. 7).

### Example 7

### Investigation of Ra Tetherin

Tetherins inhibit virus egress after dimerization and by connecting the plasma membrane and viral envelope via a coiled coil that is attached to a trans-membrane domain and GPI anchor at the opposing termini. The Ra tetherin of the present invention contains a transmembrane domain (amino acids K29-V49) but no signal peptide according to the predictive algorithms SignalP 6.0 and TOPCONS. Although signal peptides mediate translation into canonical secretory pathways they are not exclusive to all proteins that reach or transverse the plasma membrane. Here, the modification with the GPI anchor appears to be required for translocation of Ra tetherin to the lumen of the ER. A GPI attachment site is predicted by PredGPI to run from N156-A183 with N156 as ω amino acid for the anchor. The coiled-coil for dimerization is predicted by DeepCoi for amino acids L63-H150.

To investigate potential mechanisms a fortuitous SfoI restriction site was used within the last 12 bp (4 amino acids, A₁₈₀LLA₁₈₃) of the tetherin ORF for carboxy-terminal fusion to GFP. The fusion was performed by insertion of 603 bp containing tetherin out of the synthetic construct via BglII and SfoI into BglII and AgeI sites of pEGFP-N1 (Clontech, now Takara). Klenow enzyme was used on the vector cut with AgeI to generate a compatible blunt end for ligation to SfoI. The bioinformatics algorithms for prediction of membrane properties were also applied to the tetherin-GFP fusion protein. The transmembrane region and coiled-coil were highlighted also in the fusion protein, but no additional such domains. A GPI-anchor was not detected in the fusion protein. This construct is therefore expected to destroy one of the two anchors necessary for the function as a membrane to envelope linkage. However, there are few reports that internal placement of the GPI signal can be processed by the cells with reduced efficiency. The resulting plasmid (V9 pTethGFP) was used to generate a shuttle plasmid for DS IV to test whether a recombinant MVA that expresses the fusion protein can be rescued. Again, without repression a functional tMVA could not be rescued suggesting that the tetherin to EGFP fusion protein still is active or that the mechanism of interference goes beyond the properties that are currently assigned to tetherin.

### Example 8

### Cloning of the Shuttle Plasmid to Exchange D9R and D10R in MVA-CR19 Against g5R of ASFV

ASFV and vaccinia viruses are the only known viruses that reduce accumulation of dsRNA (a potent trigger of innate immunity) with decapping enzymes. The viral decapping enzymes are encoded by D9R and D10R in vaccinia virus, and by D250R in ASFV. In MVA the genes are also called MVA106R and MVA107R. In Genbank Accession number KY633487 (version KY633487.1) the position of the coding sequence for MVA106R is 110634-111275 and for MVA107R is 111272-112018. Their activity channels dsRNAs into the cellular Xrn1-mediated decay pathways. The decapping enzyme of ASFV may even preferentially attack mRNAs important for innate immunity and may thus prolong expression of transgenes. The decapping enzyme of ASFV appeared to be a good target for investigating optimization of the transgene expression pattern and to achieve an attenuated phenotype that can be used in gene swap protocols for the rapid generation of recombinants. In Genbank Accession number MN630494 (version MN630494.2) the position of the coding sequence of D250R is 133777-134529.

Replacement of D9R and D10R in MVA against ASFV D250R was performed by homologous recombination in CR.pIX cells that were infected with MVA-CR19 and transfected with a shuttle plasmid. The shuttle plasmid was cloned by stepwise assembly. First, the left and right recombination flanks were obtained by PCR with DNA isolated from MVA-infected cells. To obtain DNA, 80 μL of complete cell lysate was mixed with 20 μL of QuickExtract DNA Extraction Solution 1.0 (Epicentre, USA) and heated to 65° C. for 10 min and to 98° C. for 5 min. 4 μL of this preparation was subjected to PCR in a final volume of 25 μL with 0.15 μL of a proofreading polymerase (Qiagen, Germany), 200 nM each primer, and 125 μM each nucleotide. Thermocycling was initiated with 94° C. for 80 s, followed by 35 cycles of 94° C. for 20 s, 55° C. for 20 s and 72° C. for 90 s, and terminated with 72° C. for 5 min.

The left flank was amplified with primers SEQ ID NO 20 and SEQ ID NO 21. The forward primer is situated close to a convenient PciI site, the reverse primer contains a BglII target site. The amplicon was digested with these two restriction enzymes to obtain a fragment of 832 bp that was inserted into the same sites of a shuttle plasmid for deletion site III (derivation of this plasmid was described earlier (Jordan et al. 2019)).

The right flank was amplified with primers SEQ ID NO 22 and SEQ ID NO 23, digested with NotI and DraIII to obtain a fragment of 871 bp that was inserted into the same sites of the shuttle plasmid that already contained the left flank.

The left flank reverse primer also contained a point mutation to destroy the native ATG initiator codon of D9R to allow initiation of translation at the authentic ATG if the transgenes that are to be inserted under control of the D9R early promoter in MVA.

The DNA for insertion of the ASFV D250R gene was obtained via gene synthesis (GeneArt) using the Genbank MK128995 sequence. This DNA was designed manually to have all internal BglII, NotI, NdeI and KpnI sites deleted. This synthetic DNA was further designed to be trailed by a 275 bp repeat of the left side of the right flank, followed by an artificial Early/Late promoter. Insertion of the synthetic DNA coding for g5R was done with terminal BglII and KpnI sites in the new D9/D10 shuttle vector. This step maintains the GFP gene as a transient marker flanked by a 275 bp repeat.

Primers used for testing of successful and complete substitution are SEQ ID NO 24 and SEQ ID NO 25.

Because the two decapping enzymes are essential for vaccinia replication, g5 of ASFV must be capable of replacing the combined activities of D9 and D10, and g5R must be expressed as soon as the genes for D9 and D10 are deleted. As D9 is expressed early in the infectious cycle the promoter of this gene was used for expression of g5R. The two flanks of the shuttle plasmid for the substitution included the sequences upstream of D9R up to the ATG, and sequences downstream of D10R, starting 88 bp upstream of the stop codon. An expression cassette for GFP was included downstream of g5R to facilitate recovery of recombinants (initial experiments without this aid failed). As described above, the reporter was preceded by an internal repeat of 275 bp of the downstream recombination flank. This design allows identification of recombinants by EGFP expression and by presence of a large amplicon in PCR. The EGFP expression is not desired in a final plaque-purified clone. Having EGFP flanked by homologous 275 bp causes a deletion of the marker after several passages. The final substitution shuttle plasmid was called P22 pASFVxD9D10. Based on the MVA-CR19 backbone a recombinant called MVA-G5R was recovered as described in Example 9.

### Example 9

### Substitution of D9 and D10 Against g5R in MVA-CR19

For recombination, adherent cultures of 1e6 CR.pIX cells per well of a 6-well-plate were infected with MOI of 0.01 of MVA-CR19 and transfected with 2 μg of P22 pASFVxD9D10 plasmid 90 minutes after infection. Transfection was performed with Effectene® Transfection Reagent (Qiagen) according to the manufacturer's description, here furthermore adjusted to 200 μL buffer, 16 μL enhancer and 20 μL effectene. The complete lysate of this recombination reaction (passage 0, P0) was applied to a fresh culture of CR.pIX cells in a 6 well plate. Methylcellulose (Sigma) to 1% was applied after 3-5 h to prevent diffusion of progeny virus and to facilitate subsequent plaque picking. Plaques were identified by fluorescence microscopy and picked 48-72 h post infection by aspiration of approx. 20 μL volumes with a 100 μL pipette tip. The methylcellulose column was transferred to 500 μL of medium, sonified, and used to infect fresh monolayers in 6-well plates at 5-fold to 1000-fold dilutions for the next, methylcellulose-assisted round of plaque picking. In a typical plaque purification passage, 15-40 plaques were picked and characterized by PCR.

PCR was performed with total DNA isolated out of 4 μL of the resuspended plaques. The 4 μL were mixed with 1 μL of QuickExtract DNA Extraction Solution 1.0 (Epicentre, USA), incubated at 65° C. for 10 min and at 98° C. for 5 min.

A typical reaction in a volume of 25 μL in 1×PCR reaction buffer (Qiagen, Germany) contained 0.15 μL Taq polymerase (Qiagen), 200 nmol/L each of primers SEQ ID NO: 24 and SEQ ID NO: 25, and 125 μmol/L each nucleotide. After pre-incubation at 94° C. for 90 s, 35 cycles were performed with 94° C. for 20 s, 55° C. for 20 s and 72° C. for 140 s.

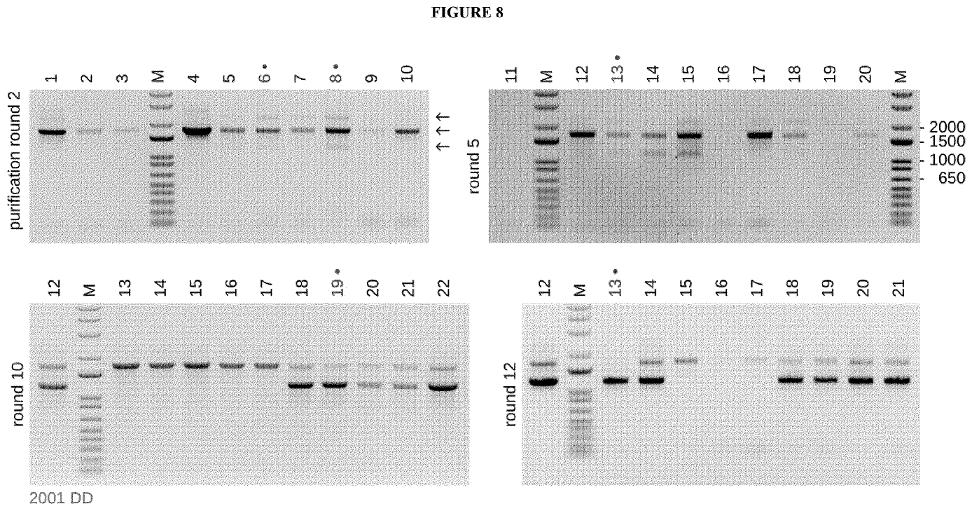

A significant experimental hurdle that had to be overcome was the prolonged presence of parental virus. The persistence of MVA-CR19 is caused by the fact that this virus now is a helper virus for MVA-G5R that (by design) is characterized by an impaired replication cycle due to the engineered mismatch in at least one defensive pathway. A total of 12 purification rounds, occasionally augmented by blind passaging (expansion or split of infected cells without intervening lysis), were required that involved characterisation of more than 260 plaques. The series of plaque purifications leading to MVA-G5R is shown in FIG. 8. The majority of successful isolations in the initial rounds resulted in three bands, MVA-G5R still containing the EGFP transient marker (2296 bp), MVA-CR19 contamination (1742 bp) and the desired MVA-G5R vector without transient marker (1219 bp). A rare preparation of MVA-G5R without parental contamination was obtained in isolation 13 of round 12.

Rescue of MVA-G5R included passages of infected cells. To address the formal possibility that D9 or D10 expression could have been restored by illegitimate recombination of MVA-G5R with wildtype MVA-CR19 we also performed diagnostic PCR intended to detect D9R or D10R coding sequences at unexpected positions in the genome. The smaller signals internal to either D9R or D10R were visible in the wildtype reference but not in MVA-G5R.

To our knowledge, MVA-G5R is the first example where one essential function of a nucleocytoplasmic large DNA virus has been replaced with the essential function of a virus of the same phylum but different family.

### Example 10

### Further Characterisation and Functionalisation of MVA-G5R

MVA-G5R causes smaller plaques in adherent CR.pIX cells, replicates to slightly lower titers and not as rapidly as the parental MVA-CR19. MVA-G5R has maintained the expected host cell restriction and cannot replicate in human HEK 293 or porcine PK-15 cells. Maintenance of attenuation is an highly important property. That an exchange of poxviral and asfariviral homologs is possible is shown here for the first time. Such an exchange is possible also into the other direction (D9 and D10 to replace g5) to facilitate generation of ASFV recombinants.

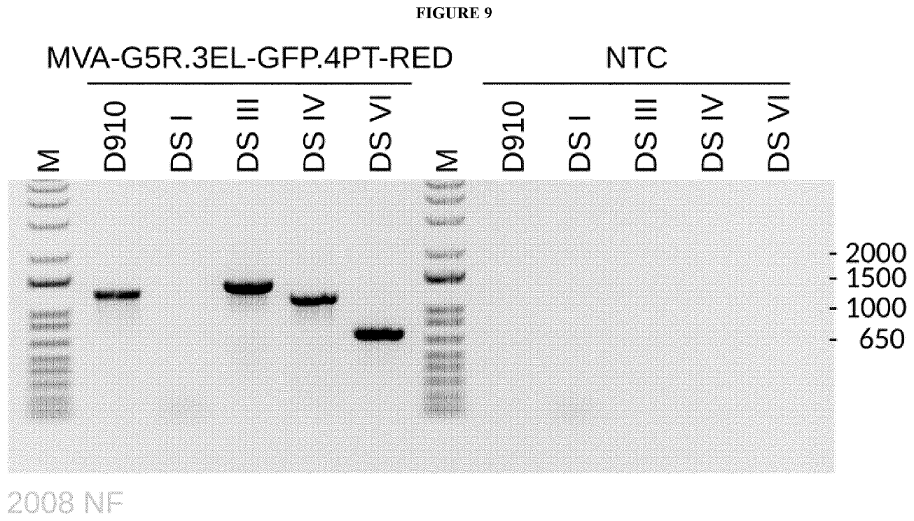

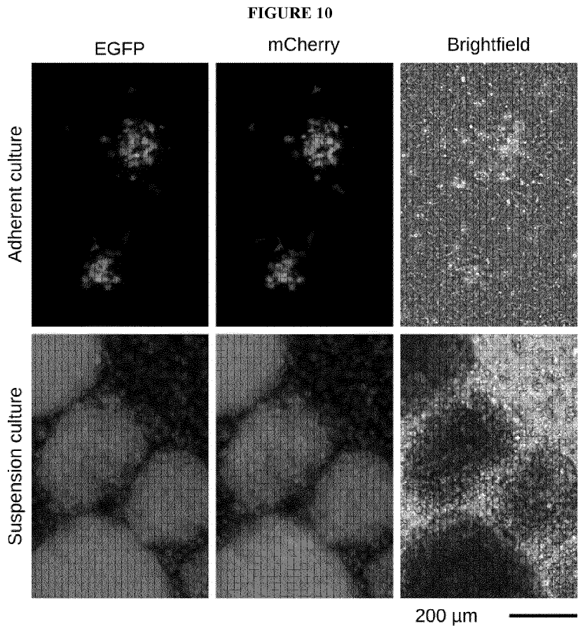

MVA-G5R represents a remarkable and novel genotype. To demonstrate that this virus remains viable and amenable to further genetic manipulation EGFP and mCherry reporter genes were recombined into DS III and DS IV. The flanks for the shuttle vectors used for this study are described in Example 1. Here, the coding sequence and mH5 promoter to drive expression of EGFP were cloned into the shuttle vector for DS III and the coding sequence and P11 promoter to drive expression of mCherry were cloned nto the shuttle vector for DS IV. The method for recombination is described in Example 9. Generation of a version of MVA-G5R that expresses both reporters was done by sequential recombination, first into DS III and then into DS IV. The intended substitution with g5R, presence of markers characteristic for MVA-CR19 and stable insertion of the two reporter proteins at the two different genomic sites was confirmed by multiple PCR reactions (FIG. 9). Viability also of a functionalised virus was demonstrated via expression of EGFP and mCherry in adherent monolayers of CR.pIX cells. That MVA-G5R can also be produced in a GMP-conform process in chemically-defined medium was demonstrated via expression of the two reporters in suspended CR.pIX cell aggregates (FIG. 10).

A drastic attenuation has been achieved with the replacement of both, D9 and D10, against the single decapping enzyme of ASFV. Replication of MVA-CR19 is superior to replication of MVA-G5R. Retrieval of MVA-G5R required careful isolation processes for complete removal of the parental helper virus. As described for tetherin, the novel insertion site D9/D10 can, thus, be considered to be functionalized for a rapid gene swap. This rapid gene swap is achieved with the here described pASFVxD9D10 shuttle plasmid furthermore equipped thus that the gene of interest is flanked by a functional D9R as a left flank and a functional D10R as a right flank. The D250R gene encoding g5R may or may not be contained. Recombination restores the parental genotype for improved replication together with the novel vectored gene of interest. Additional designs can be achieved by using only one gene for the decapping enzymes, for example D9R, and maintaining the D250R gene in the shuttle plasmid. Conversely, the D250R gene can be maintained and D10R is provided together with a gene of interest. Based on the work described here, any combination of D9R, D10R and/or D250R is conceivable.

Use of MVA-G5R furthermore includes use as a component for a vaccine against ASFV and as an oncolytic agent in animal and human immunotherapy. The mosaic virus that contains D250R in the poxviral context is predicted to cause a delayed yet greater activation of innate immunity which is beneficial to direct the immune system against intended targets such as tumors. Because innate immune responses are delayed in some cells the vector can express the recombinant vaccine epitopes to higher levels for longer intervals. Because induction and progression of programmed cell death at later stages of the infection still is possible beneficial inflammatory co-stimulation at sites of antigen presentation and immune cell engagement are still supported.

### Example 11

Transfer and Demonstration of the Selection System with a Tetherin from a Haematophagous Bat

To demonstrate that the selection system is not restricted to one particular tetherin, a second set of experiments has been performed with the ortholog of a distant species of the Egyptian fruit bat. The taxonomic order of the bats (Chiroptera) is the second largest order of mammals and divided into two suborders, Pteropodiformes and Vespertilioniformes. Frugivorous megabats such as the Old-World Rousettes are in the former suborder whereas most echolocating microbats are in the second suborder. The New World hematophagous bats of the Vespertilioniformes are unique in their metabolic adaptation for feeding on blood of mammals and birds, and possibly in their adaptation to exposure of blood-borne pathogens from their vertebrate preys. The tetherin sequence from the common vampire bat (Desmodus rotundus, abbreviated “Dr”) was obtained from Genbank with accession number XM_024561004.3. The coding sequences comprise 552 bp for Ra (Rousettus aegyptiacus) and 666 bp for Dr, respectively. Only 43% of the amino acids are identical (and 56% are similar) between the two proteins. As described above for Ra, Dr tetherin was also optimized for human codon frequency and absence of poxviral sequences that may interfere with transcription (Yang et al., 2011, PMID 21490097; Yuen et al., 1987, PMID 3476956). A synthetic DNA (GeneArt) of the optimised Dr sequence was inserted into shuttle vectors for deletion sites IV, V, VI and the TK locus. The Dr tetherin DNA sequence was furthermore designed to start with CTG TAC AAG ggc gcc, where TGTACA is a BsrGI site that is also contained at the 3′ end of the coding sequence for EGFP. This arrangement allowed fusion of the green fluorescent reporter to the N-terminus of tetherin for investigation of tetherin function and subcellular distribution.

tMVA recombinants that conditionally express Dr tetherin were generated in tCR9 cells as described in Example 3. Variations of recombinant viruses that were generated for the characterisations described in the examples below include tMVA that expresses Dr tetherin and mCherry out of DS VI (tMVA-CR19.6HYBdxT-Vamp-PT 1-RED), a virus containing the GFP-tetherin fusion (tMVA-CR19.6HYBdxT-GFPVamp-PT1-RED), and a virus that contains a tetherin copy each in different insertions sites, including a virus that contains Dr and Ra tetherins (MVA-CR19.6HYBdxT-Teth-P11BLUE.TKHYBdxT-Vamp-P11-RED). The DNA sequence for the Dr-tetherin fusion construct under control for the Hyb-tetO promoter is shown in SEQ ID NO: 28 from the BsrGI site to Dr tetherin stop codon.

### Example 12

Tetherin from the Vampire Bat is an Efficient Selection Marker.

On the day prior to infection, 5e5 adherent tCR9 cells were seeded per well of a 12-well plate. They were infected on the following day with an MOI of 0.01 with CR19 viruses that express EGFP only (as control), Ra tetherin, Dr tetherin, a GFP-Dr-tetherin fusion protein, or both Ra and Dr tetherins. EGFP was expressed out of DS III, all tetherins and mCherry out of DS VI with exception of the tetherin double-positive virus that expressed the Ra tetherin together with mTagBFP2 out of DS VI and Dr tetherin together with mCherry out of the TK location. All tetherins were under control of the hybrid tetO-equipped promoter, mCherry was under control of the late P11 promoter, EGFP of the reference virus was under control of the standard (non-switchable) hybrid promoter. Doxycycline was added to 1 μg/mL to half of the wells at the time of infection to de-repress tetherin expression.

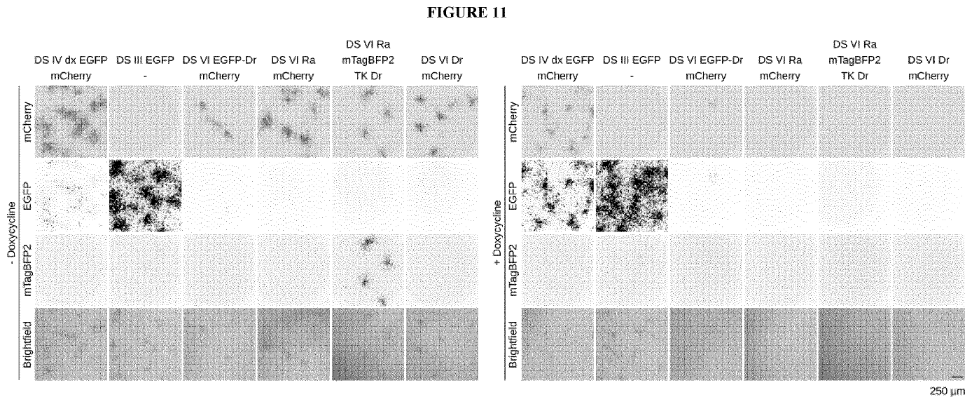

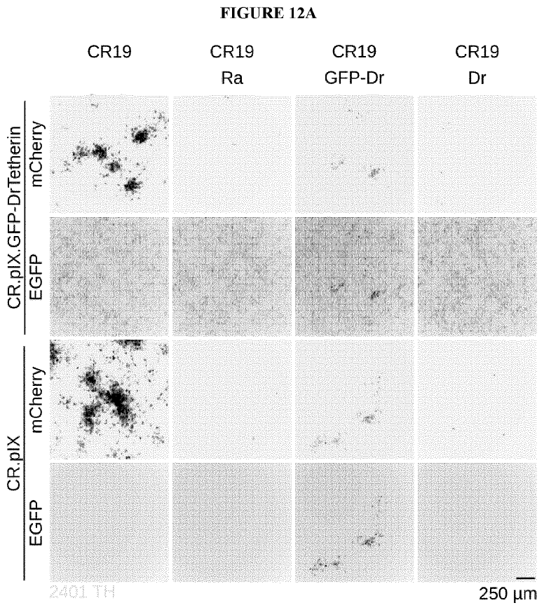

As expected and shown in FIG. 11, addition of doxycycline to allow expression of either Ra or Dr tetherin abolished replication of the respective viruses. The MVA-CR19 positive control that expresses EGFP out of DS III and does not express tetherin was no affected by addition or absence of doxycyclin. Another control virus, MVA-CR19 that contains a dual expression cassette with EGFP under control of the switchable Hyb promoter and mCherry in anti-parallel direction under control of the constitutive P11 promoter gave a fascinating and consistent pattern dependent on doxycycline addition: if expression of EGFP is inhibited in tCR9 cells then expression of the neighboring mCherry cassette is strong. If EGFP expression is deblocked then mCherry expression is weaker, probably due to interference by a readthrough by the EGFP transcript into the mCherry cassette. Independent of EGFP inhibition or not, MVA-CR19 with the dual expression cassette but without tetherin replicates as expected in presence or absence of TetR or doxycycline. This experiment demonstrates that doxycycline by itself has no effects on viral replication and that all described effects are mediate by tetherin. A virus that expresses both tetherins from different insertion sites of its genome was also impaired (FIG. 11) and will be discussed in greater detail in Example 13. The virus that expresses a Dr tetherin fused at the amino terminus to EGFP was also impaired in replication. That only spotty and highly infrequent GFP fluorescence could be observed also in presence of doxycycline is a visible control for tetherin efficacy.

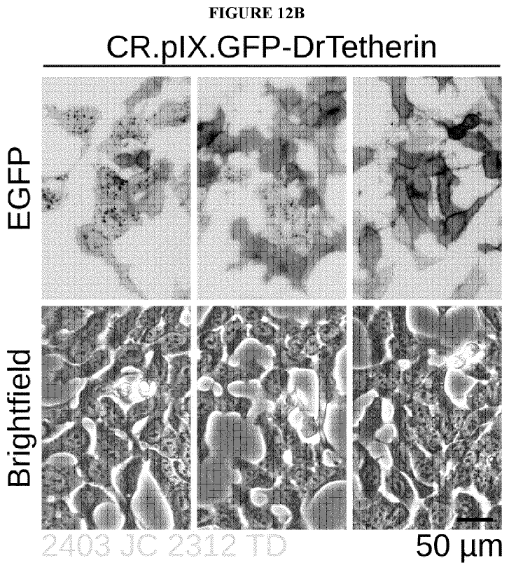

In a complementary set of experiments, and to demonstrate a correct design of the fusion protein, GFP-tetherin was cloned for constitutive expression from a CMV promoter and stably transfected into CR.pIX cells. As shown in FIG. 12 A these cells limit replication of normal MVA, although not to levels seen with tMVA (compare panels with infection by a CR19 virus that expresses mCherry but no tetherin). Viruses that express tetherin (either Ra, Dr or a GFP-Dr-tetherin fusion) are strongly inhibited. The virus that expresses the GFP-Dr-tetherin fusion appears to be inhibited to a lower degree compared to the other viruses.

As shown in FIG. 12 B, distribution of the EGFP-Dr-tetherin appears to be in two major cellular compartments, at the surface at the plasma membrane and in intracellular spots that may be localised to the perinuclear organelles. In summary, the results obtained with the stable cell line suggests that (i) the chiropteran tetherin by itself is not toxic to the cell line, (ii) that a GFP-tetherin fusion protein distributes not only to the plasma membrane but can be found also on intracellular structures, (iii) that tetherin interferes with poxviral replication also when provided in trans (outside of the viral genome), and (iv) that the GFP-tetherin fusion protein is attenuated and thus allows to adjust the selection strength against non-recombinants.

### Example 13

### Concurrent Rapid Replacement of Two Insertions Sites

One of the advantages of poxviral vectors is their capacity to stably maintain several large transgene expression cassettes. However, insertion of transgenes at different sites in the viral genome requires sequential insertions and intermittent plaque purification. Considering that a single recombination already benefits from the rapid selection system described here, a dual insertion further enhances generation of recombinant viruses significantly. As a first step, it was first demonstrated that generation of a virus that expresses two tetherins out of different positions in the genome is possible. This demonstration is necessary and not trivial because even distant repeats of tetherin in the genomic core may be deleted by homologous recombination and because dose effects may interfere with insertions of more than one copy (beyond the dual insertion in the telomeric DS IV or CR19). It was observed in the present invention that both insertion of two copies of Ra tetherin and of the tetherins from different species (Ra and Dr) was possible. Triple and higher repeats may also be possible with appropriately designed, for example attenuated versions of tetherin such as the above-described fusion to EGFP.

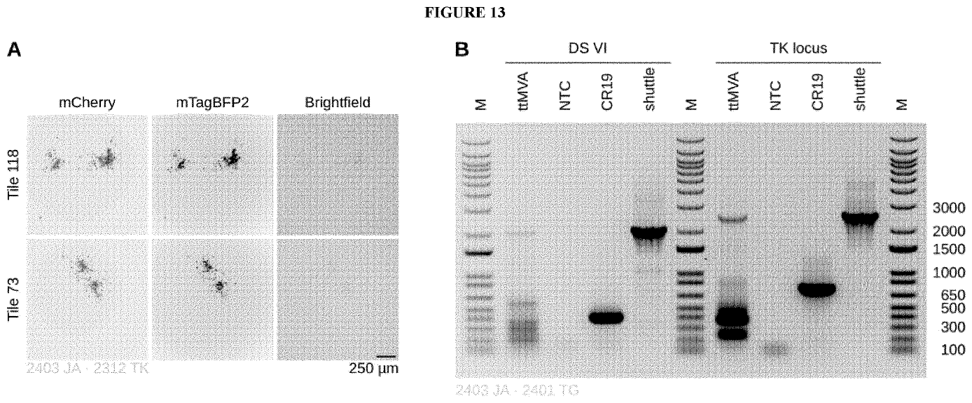

One of the viruses with two tetherin inserts is MVA-CR19.6HYBdxT-Teth-P11BLUE.TKHYBdxT-Vamp-P11-RED that encodes Ra tetherin in DS VI and Dr tetherin in the TK site. For facilitated interpretation of results, expression out of DS VI is marked with the mTagBFP2 blue fluorescent protein and out of the TK site with the mCherry reporter. Generation of this virus was done stepwise, first generating MVA-CR19.6HYBdxT-Teth-P11BLUE (23T11-AG) using blue fluorescence to guide plaque purification. That virus was recombined with the shuttle plasmid pTKHYBdxT-Vamp-P11-RED (Y6). Purification of the dual-tetherin virus (designated ttMVA) was performed by selection of blue and red fluorescence. FIG. 13 B shows the genetic confirmation for one of three independent plaques. Figure FIG. 13 A shows four separate typical plaques of the ttMVA with red and blue fluorescence.

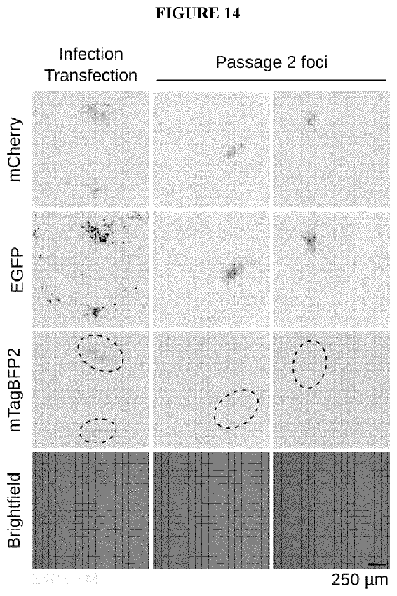

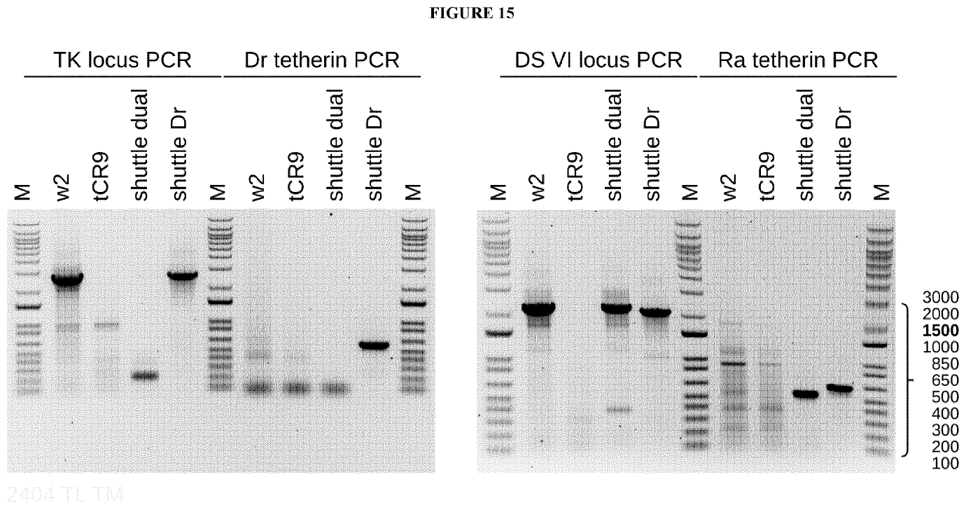

This ttMVA was used to infect tCR9 cells in a well of a 6-well plate in 2 mL of culture medium with an MOI of 0.01 followed by simultaneous transfection with 1 μg each of two shuttle plasmids for recombination of a dual expression cassette coding for EGFP and mCherry into DS VI and the TK site. Medium was replaced 4 h post transfection. The recombination reaction was imaged and harvested 48 h post infection. Infection for passage 1 was performed with 100 μL to 300 μL of the lysate. Medium was replaced after 2 h and doxycycline was added to 1 μg/mL. The culture was imaged and harvested 72 h post infection. Passage 2 was performed similarly with 500 μL of the lysate from the previous passage, followed by PCR analysis to demonstrate successful gene swapping at both sites (DS VI and TK; shown in FIG. 14. The cassette containing the blue-fluorescent protein has been replaced and the virus replicates normally on CR.pIX cells without dependence on TetR repression. Expected amplicon sizes with primer pairs screen-VI (SEQ ID NO: 15 and SEQ ID NO: 16) are 2030 bp (expression cassette with P11-mCherry and hybrid-tetO-Ra tetherin (W6)), 2035 bp (P11-TagBFP2 and hybrid-tetO-Ra tetherin (W76)), 2195 bp (mH5-EGFP and P11-mCherry, p65T-dual (Q31)), and 406 bp (for native, non-loaded DS VI). Expected amplicon sizes with primers Li_FL_TK_for and TK_RF_for (SEQ ID NO: 26 and SEQ ID NO: 27) are 2414 bp (hybrid-tetO-Ra tetherin and P11-mCherry (W34)), 2591 bp (mH5-EGFP and P11-mCherry (U53)), 2525 (P11-mCherry and hybrid-tetO-Dr tetherin (Y6)) and 754 bp (for native TK site). As shown in FIG. 15, the appearance of the novel expression cassette that replaces tetherin is also visible in a PCR reaction, concurrent with loss of parental tetherins. This experiment demonstrates that a simultaneous replacement towards double-recombinant viruses can be performed without plaque picking within 2 passages.

### Example 14

### Rapid Recombination and Rescue in Suspension Cells

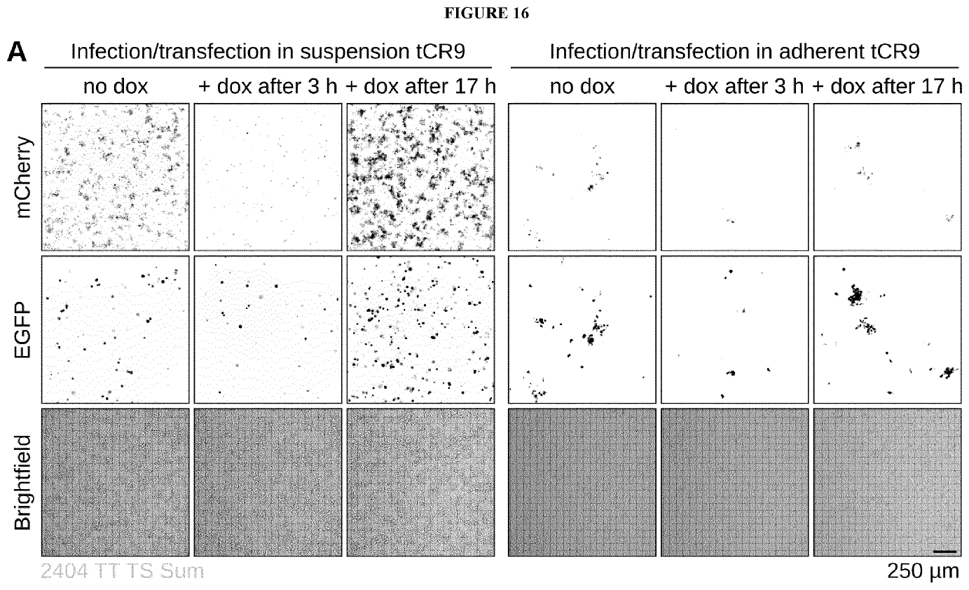

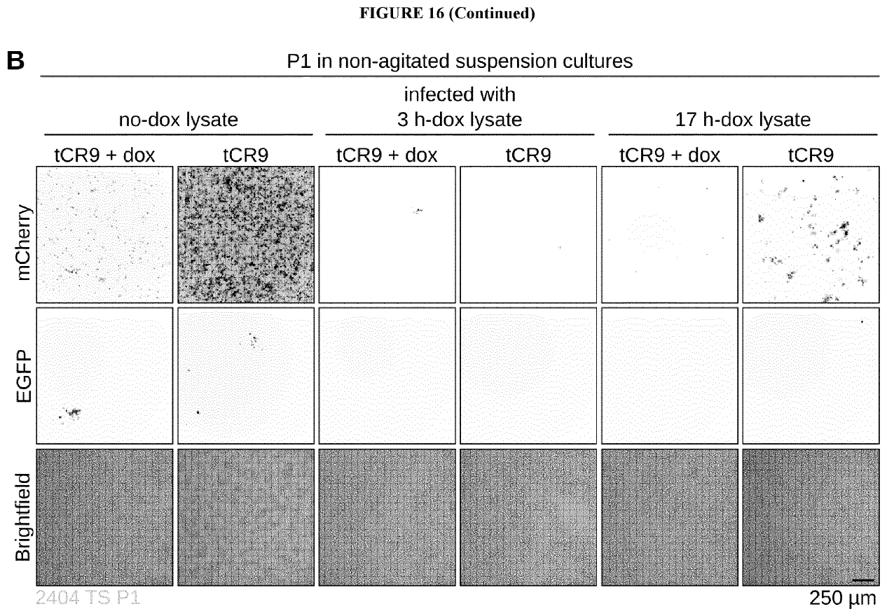

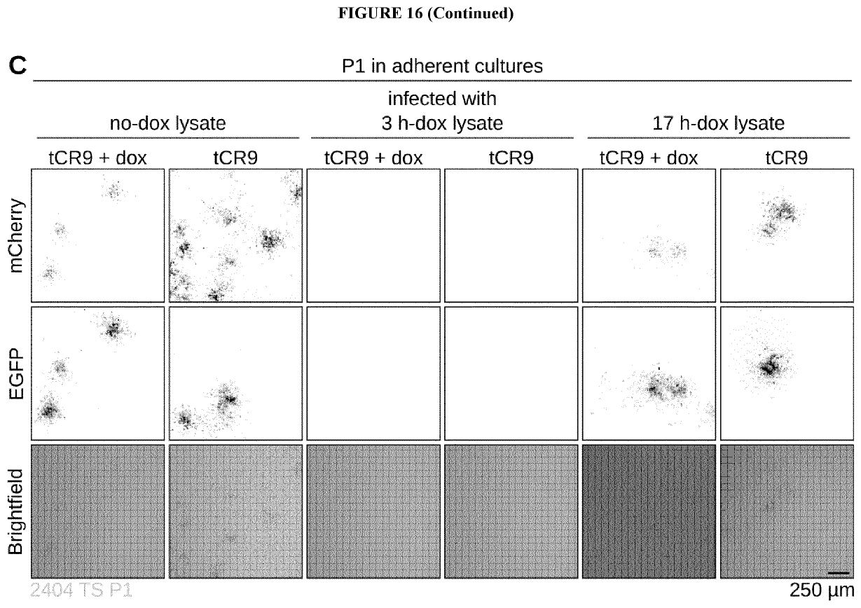

Current technologies for generation of recombinant viruses require access to plaques or foci for sequential isolation and purification steps. They furthermore require that recombinant viruses can be visually distinguished from non-recombinant viruses. The advantage of the stringent selection system against parental viruses described here is that generation, rescue and purification of recombinants can also be performed in suspension cultures without isolation by automated cell sorting or single-cell cloning. To demonstrate such an approach, transfection was scaled to the required small volumes for amplification of vectors after recombination: on the day of the transfection, 0.5e6 cells in 1 mL volume were infected with appropriate tMVA to an MOI of 0.05 and transferred into one well of a 12-well plate. Within the next 90 minutes, 1 μg of shuttle plasmid was resuspended in 50 μL of non-supplemented DMEM (Gibco; corresponding to 5% of the total culture volume). FectoVIR®-AAV (Polyplus Sartorius) was vortexed briefly and 2 μL therefrom were added to the DNA suspension. The transfection mix was mixed vigorously and added to the infected culture after incubation for 30 minutes at room temperature. The infected/transfected culture was kept without further agitation in a stationary incubator for 48-72 h. Lysis for the next selection and amplification steps was done by ultrasonification as described above. Virus obtained in the first passage after infection/transfection was characterised by expression of fluorescence. The first step, infection and transfection in suspension is shown in FIG. 16 (A) with MVA-CR19.4HYBdxT-Teth-RED that contains Ra tetherin and mCherry in DS IV. Recombination was performed with shuttle plasmid p45T-Dual (Q30) for DS VI that expresses EGFP and mCherry. Maintenance of mCherry expression is indicative of legitimate homologous recombination and expression of EGFP is indicative for replacement of the tetherin selection marker. As a comparison to previous results and to demonstrate stringency, the experiment was performed in parallel in adherent tCR9 cells. Both threads were furthermore performed with or without release of tetherin expression during the recombination (passage 0, P0) in panel (A) and during the selection process at passage 1 (FIG. 16 (B) for suspension cells and (C) for adherent cells). The results confirm that tetherin expression should be suppressed at least transiently during recombination (no desired recombinant vectors or productive infection with parental tMVA viruses were observed if dox was added 3 h post infection). The results also confirm that in suspension and adherent cells, the desired recombinant viruses with dual expression of EGFP and mCherry are obtained only if tetherin expression is allowed by addition of dox to tCR9 at passage 1 (or by amplification in cells that do not express tetR protein).

### REFERENCES

Beissert T, Koste L, Perkovic M, Walzer K C, Erbar S, Selmi A, Diken M, Kreiter S, Tureci O, Sahin U (2017) Improvement of In Vivo Expression of Genes Delivered by Self-Amplifying RNA Using Vaccinia Virus Immune Evasion Proteins. Hum Gene Ther 28:1138-1146. https://doi.org/10.1089/hum.2017.121
Burgess H M, Pourchet A, Hajdu C H, Chiriboga L, Frey A B, Mohr I (2018) Targeting Poxvirus Decapping Enzymes and mRNA Decay to Generate an Effective Oncolytic Virus. Mol Ther Oncolytics 8:71-81. https://doi.org/10.1016/j.omto.2018.01.001
Chakrabarti S, Sisler J R, Moss B (1997) Compact, synthetic, vaccinia virus early/late promoter for protein expression. BioTechniques 23:1094-1097
de Freitas L F D, Oliveira R P, Miranda M C G, Rocha R P, Barbosa-Stancioli E F, Faria A M C, da Fonseca F G (2019) The Virulence of Different Vaccinia Virus Strains Is Directly Proportional to Their Ability To Downmodulate Specific Cell-Mediated Immune Compartments In Vivo. J Virol 93. https://doi.org/10.1128/JVI.02191-18
Jordan I, Horn D, John K, Sandig V (2013) A Genotype of Modified Vaccinia Ankara (MVA) that Facilitates Replication in Suspension Cultures in Chemically Defined Medium. Viruses 5:321-339. https://doi.org/10.3390/v5010321
Jordan I, Horn D, Oehmke S, Leendertz F H, Sandig V (2009) Cell lines from the Egyptian fruit bat are permissive for modified vaccinia Ankara. Virus Res 145:54-62. https://doi.org/10.1016/j.virusres.2009.06.007
Jordan I, Horn D, Thiele K, Haag L, Fiddeke K, Sandig V (2019) A Deleted Deletion Site in a New Vector Strain and Exceptional Genomic Stability of Plaque-Purified Modified Vaccinia Ankara (MVA). Virol Sin. https://doi.org/10.1007/s12250-019-00176-3
Liu S-W, Katsafanas G C, Liu R, Wyatt L S, Moss B (2015) Poxvirus decapping enzymes enhance virulence by preventing the accumulation of dsRNA and the induction of innate antiviral responses. Cell Host Microbe 17:320-331. https://doi.org/10.1016/j.chom.2015.02.002
Parrish S, Moss B (2006) Characterization of a vaccinia virus mutant with a deletion of the D10R gene encoding a putative negative regulator of gene expression. J Virol 80:553-561. https://doi.org/10.1128/JVI.80.2.553-561.2006
Sliva K, Resch T, Kraus B, Goffinet C, Keppler O T, Schnierle B S (2012) The cellular antiviral restriction factor tetherin does not inhibit poxviral replication. J Virol 86:1893-1896. https://doi.org/10.1128/JVI.05198-11
Wang E, Petrakova O, Adams A P, Aguilar P V, Kang W, Paessler S, Volk S M, Frolov I, Weaver S C (2007) Chimeric Sindbis/eastern equine encephalitis vaccine candidates are highly attenuated and immunogenic in mice. Vaccine 25:7573-7581. https://doi.org/10.1016/j.vaccine.2007.07.061
Wyatt L S, Shors S T, Murphy B R, Moss B (1996) Development of a replication-deficient recombinant vaccinia virus vaccine effective against parainfluenza virus 3 infection in an animal model. Vaccine 14:1451-1458. https://doi.org/10.1016/s0264-410x(96)00072-2
Yang Z, Bruno D P, Martens C A, Porcella S F, Moss B (2011) Genome-wide analysis of the 5′ and 3′ ends of vaccinia virus early mRNAs delineates regulatory sequences of annotated and anomalous transcripts. J Virol 85:5897-5909. https://doi.org/10.1128/JVI.00428-11
Yuen L, Moss B (1987) Oligonucleotide sequence signaling transcriptional termination of vaccinia virus early genes. Proc Natl Acad Sci USA 84:6417-6421

INFORMAL SEQUENCE LISTING

SEQ ID NO: 1: ttacatgtgactgtgttcagggtatag
SEQ ID NO: 2: catagctagccgtacatccacatct
SEQ ID NO: 3: ttagcggccgcgtacggctttagaaatgag
SEQ ID NO: 4: ttcacgtagtgggtctgaactgggcac
SEQ ID NO: 5: ttacatgtggtgcagccaatactattacag
SEQ ID NO: 6: atagctagcatgaaggctgaacgtg
SEQ ID NO: 7: tagcggccgcgtcttatagcatgaattcg
SEQ ID NO: 8: ttcacgtagtgttgcgagcattactgcg
SEQ ID NO: 9: tcacgtagtggaataccgacggcgttaatag
SEQ ID NO: 10: ttgcggccgcatcatgaatgcgtataataaag
SEQ ID NO: 11: tagctagcgataaacttaatgaaaaatgtttttcg
SEQ ID NO: 12: ttacatgtcctgtacgatgagttc
SEQ ID NO: 13 gtgctataacgcgactatctag
SEQ ID NO: 14: tgttggtagttcttccgtgg
SEQ ID NO: 15: tttggtaatggtttctcatgtgg
SEQ ID NO: 16: gacatttagtttgagtgttcctg
SEQ ID NO: 17: (552 bp)
ATGGCCCCCGCACTCTACCATTATTTCCCCGTACCTATGGATGACCATTC
AGAGAAAGTCGTGTTGAGGAACCGTAAGAT
GCTGAAGTGGTTATGGATACTGCCTGTGTTAGCTTTGGTGTTAAGTCTCC
TGGTGGCTCTCATAGTCTTTGCTATTAAAG
CCAATTCTAAAGCCTGTAAGGATGGACTGCTTGCTGAACAGAAGTGTCTT
AACAAAACTAGGCTGTTAGAACTTCAGCTG
ACCCAGGCTCAAGAATCTTTGGTGGTGGCTGAAGCTCAAGCATACCTGTG
TAATCAGACTGTAGGCACCCTGAAAAGCTC
TCTGGAAATGGAGAAGGATGAGTCTCAGAAACAAAGGGAACTGGCTCAGA
AACTGCAGGGAGAAAACGGTAATCTGAAAC
AGGAACTGGAAAACATGATGGCCGAGCTTGAGCAGCTGCGGAAAGAACAC
GCCTCGGATGAAAAGAACGGCTCTACTAGC
TCTAGGAATGCAAGGAGTTTCTTAGTTGTGGCAGTCTTACTCAGCCTGTC
TTTTGGCGCCCTACTCGCATGA
SEQ ID NO: 18:
TAAAAAT TGAAAATAAA TACAAAGGTT CTTGAGGGTT
GTGTTAAATT GAAAGCGAGA AATAATCATA AATAGGATCT
ATCCCTATCA GTGATAGAGA TCTCCCTATC AGTGATAGAG
ATCAGATCTC GAGCTCAACC CGGGAACTCT GCAGTCGACG
GTACAGGTCG CCACCGGATC CTGTTAACCC ATG
SEQ ID NO: 19:
A AAAATTAAAA TTTTATTTTT TTTTTTTGGA ATATAAATAA
GATCTATCCC TATCAGTGAT AGAGATCTCC CTATCAGTGA
TAGAGATCAG ATCTCGAGCT CAACCCGGGA ACTCTGCAGT
CGACGGTACA GGTCGCCACC GGATCCTGTT AACCCATG
SEQ ID NO: 20: ttctaatggaggctcaacaag
SEQ ID NO: 21: acatAGATCTcccaGactaagagctatttttaaac
SEQ ID NO: 22: tataGCGGCCGCatatgctctagatactgcaaaac
SEQ ID NO: 23: ttaaCACGTAGTGttagcatcccatggtaaatgtc
SEQ ID NO: 24: GTCTAACGGCTTCAATCG
SEQ ID NO: 25: GTCCGGTTGGAAAACCTT
SEQ ID NO: 26: CTCTCTAGCTACCACCGCAA
SEQ ID NO: 27: ACGGTTTATCTAACGACACAACA
SEQ ID NO: 28:
CTGTACAAGg gcgccATGAC GTCTACATTT TACCACTACA
TCCCCCTACC AATGTCTGAG AACTCCCGCG AGCTTATGTT
GGAATACCAT AAACTCCCGA GATGGCTGGG TATCCTCCTA
GCGCTCCTGG TGCTTCTGGT
AGTGGGGCTG CTTGTGGCTA CGATTATCCT GGCCGTGCAG
GCTCATTCAC
CTGCCTGCAA GGACGGTCAT CGGGCTGAGC AGGAATGCAG
GAACTTTACT CACTTATTAG
AGAGTCAAAG AACCAGAGCA CAGGAGATCC TCTTAAAAAC
TAAAGCCCAG GCTGCCACAT
ATAATCAGAC GGTGGTCACT TTGATGGCAT
CGCTGAAAGT AGAGCAGGCA CAGGGACAGA AGCTGCGCGA
ACAAGTGCAG GAACTGCAAG AAGAGATCAA GACTCTTAAG
CAAAAATTAC AGGACACAAC ACAGAAATTG CAGGTGACCA
CGACCGAACT ACAAAACACT ACTCAAAAGC
TCCAGGTTAC CACAGCTAAG CTCCAGGTGA CTACACAAAA
ACTCCAAGAT ACGACTACCG AACTGAATCA GCTGCGGAAG
GACCACGAGA GCTTTGACAG GGGTAACGGG AGCACCAGCT
CTGGGAATAC TCTGTCCCTG TCCGTGGTGG
TCCTGCTTCT CACGCTTTCC CTTTTGGACT TGCTGGCATG A

1. An enveloped virus containing a polynucleotide comprising a/at least one nucleotide sequence encoding tetherin.

2-3. (canceled)

4. The enveloped virus of claim 1, wherein the enveloped virus is a virus whose replication is affected by tetherin.

5. (canceled)

6. The enveloped virus of claim 1, wherein the enveloped virus is a virus of the Poxviridae family.

7. The enveloped virus of claim 6, wherein the virus of the Poxviridae family is selected from the group consisting of a vaccinia virus, an avipoxvirus, and a parapoxvirus.

8. The enveloped virus of claim 7, wherein
the vaccinia virus is an attenuated vaccinia virus selected from the group consisting of a Modified Vaccinia Ankara (MVA) virus, an MVA related virus, a vaccinia virus Dairen-I, a vaccinia virus strain NYVAC,
the avipoxvirus is selected from the group consisting of a canarypox virus and a fowlpox virus, or
the parapoxvirus is an ORF virus.

9-10. (canceled)

11. The enveloped virus of claim 1, wherein the tetherin is tetherin of chiropteran, human tetherin, or avian tetherin.

12. The enveloped virus of claim 1, wherein the/the at least one nucleotide sequence encoding tetherin is under control of/operably linked to a promoter.

13. (canceled)

14. A cell line expressing a factor antagonizing tetherin.

15. (canceled)

16. The cell line of claim 14, wherein the factor antagonizing tetherin is selected from the group consisting of a Tet Repressor (TetR) modulating activity of a tetherin promoter, an siRNA against tetherin, an siRNA against the mRNA encoding tetherin, and a protein factor that interferes with tetherin post-translation.

17-18. (canceled)

19. A cell line expressing a factor antagonizing tetherin, wherein the factor antagonizing tetherin expressed in the cell line is capable of suppressing the effect of tetherin expressed by the virus of claim 1.

20. The cell line of claim 14, wherein the cell line is selected from the group consisting of an avian cell line and a mammalian cell line.

21. A method for propagating an enveloped virus containing a polynucleotide comprising a/at least one nucleotide sequence encoding tetherin comprising the step of: culturing the enveloped virus containing a polynucleotide comprising a/at least one nucleotide sequence encoding tetherin in a cell line expressing a factor antagonizing tetherin.

22. (canceled)

23. The method of claim 21, wherein
the enveloped virus is a virus whose replication is affected by tetherin, and/or the factor antagonizing tetherin is selected from the group consisting of a Tet Repressor (TetR) modulating activity of a tetherin promoter, an siRNA against tetherin, an siRNA against the mRNA encoding tetherin, and a protein factor that interferes with tetherin post-translation.

24. (canceled)

25. A method for producing a virus population comprising a recombinant enveloped virus (containing a polynucleotide comprising a nucleotide sequence of interest) comprising the steps of:
(i) transfecting a cell line which
(a) expresses a factor antagonizing tetherin and
(b) comprises an enveloped virus containing a first polynucleotide comprising a/at least one nucleotide sequence encoding tetherin
with a second polynucleotide comprising a nucleotide sequence of interest, wherein the second polynucleotide is capable of homologous recombination with the first polynucleotide contained in the enveloped virus, and
(ii) culturing the enveloped virus in the cell line transfected in (i), thereby obtaining a virus population that is composed of a recombinant enveloped virus comprising, instead of the/the at least one nucleotide sequence encoding tetherin, the nucleotide sequence of interest, and an enveloped virus containing the first polynucleotide (comprising a/at least one nucleotide sequence encoding tetherin).

26. The method of claim 25, wherein the cell line transfected in step (i) has been infected with an enveloped virus containing a first polynucleotide comprising a/at least one nucleotide sequence encoding tetherin before step (i).

27. The method of claim 25, further comprising the steps of:
(iii) infecting a cell line not expressing a factor antagonizing tetherin with the virus population of step (ii), and
(iv) culturing the virus population, thereby
separating the recombinant virus comprising, instead of the/the at least one nucleotide sequence encoding tetherin, the nucleotide sequence of interest, from the enveloped virus containing the first polynucleotide (comprising a/at least one nucleotide sequence encoding tetherin), or
enriching the recombinant virus comprising, instead of the/the at least one nucleotide sequence encoding tetherin, the nucleotide sequence of interest.

28. The method of claim 27 further comprising the step of:
(v) isolating the virus population from the cell line, wherein the virus population is mainly composed of a recombinant enveloped virus comprising, instead of the/the at least one nucleotide sequence encoding tetherin, the nucleotide sequence of interest.

29-30. (canceled)

31. The method claim 25, wherein the cell line is selected from the group consisting of an avian cell line and a mammalian cell line.

32. A pharmaceutical composition comprising an enveloped virus of claim 1.

33. A method of treating a disease in a patient, the method comprises the steps of
(i) providing the enveloped virus of claim 1 or a pharmaceutical composition comprising the enveloped virus, and
(ii) administering the enveloped virus or the pharmaceutical composition to a patient in need thereof.

34-50. (canceled)
